(19) United States
(12) Patent Application Publication
(10) Pub. No.: US 2013/0224285 A1
(43) Pub. Date: Aug. 29, 2013

(54) ESTROGEN RECEPTOR ALPHA POLYPEPTIDE SEQUENCE, DIAGNOSTIC AND THERAPEUTIC APPLICATIONS THEREOF

(71) Applicants: Dominique Gallo, Bruxelles (BE); Guy LeClercq, Bruxelles (BE); Iman Haddad, Paris (FR); Joelle Vinh, Paris (FR); Elias Castanas, Heraklion (GR); Marilena Kampa, Heraklion (GR); Vasiliki Pelekanou, Heraklion (GR); Yves Jacquot, Paris (FR)

(72) Inventors: Dominique Gallo, Bruxelles (BE); Guy LeClercq, Bruxelles (BE); Iman Haddad, Paris (FR); Joelle Vinh, Paris (FR); Elias Castanas, Heraklion (GR); Marilena Kampa, Heraklion (GR); Vasiliki Pelekanou, Heraklion (GR); Yves Jacquot, Paris (FR)

(73) Assignees: Universite Libre de Buxelles, Brussels (BE); UNIVERSITE PIERRE ET MARIE CURIE - UPMC, Paris Cedex 05 (FR); CNRS, Paris (FR); UNIVERSITY OF CRETE - SCHOOL OF MEDICINE, Heraklion (GR)

(21) Appl. No.: 13/876,428

(22) PCT Filed: Oct. 13, 2011
(86) PCT No.: PCT/EP11/67848

§ 371 Date: Mar. 27, 2013

(30) Foreign Application Priority Data
Oct. 15, 2010 (EP) 10187761.1

(51) Int. Cl. C07K 14/72; C07K 7/08; C07K 7/06; G01N 33/68

(52) U.S. Cl. CPC C07K 14/721; G01N 33/6893; C07K 7/08; C07K 7/06
USPC 424/450; 435/7.23; 530/324; 530/325; 530/326; 530/329; 530/363; 530/350; 514/21.3; 514/21.4; 514/21.7; 536/23.5; 514/44 R; 424/185.1; 435/320.1; 435/366; 435/254.2; 435/252.33; 435/252.3; 435/252.34; 424/93.2; 424/93.21; 530/387.9; 514/19.3

(57) ABSTRACT

The present invention presents new insights in the mechanism of action of the estrogen receptor alpha in breast cancer cells and provides means and tools for modulating said mechanisms of action, thereby influencing the proliferation of estrogen-positive cells such as cancer cells.

### FIELD OF THE INVENTION

The present invention is situated in the medical field, more in particular in the field of medical diagnosis and treatment, more specifically in the diagnosis and treatment of cancers such as breast cancer, using a new diagnostic and therapeutic target peptide issued from the Estrogen Receptor alpha (ER-alpha).

### BACKGROUND OF THE INVENTION

The Estrogen Receptor alpha (ER-alpha) has been associated with a variety of cancers, including breast cancer, endometrial cancer, cervical cancer and ovarian cancer. In breast cancer, positive ER-alpha status is associated with favorable prognostic attributes including a lower rate of cell proliferation and histologic evidence of tumor differentiation. During the first several years following diagnosis, patients having ER-alpha-positive tumors tend to have a lower recurrence rate, but this is balanced by a higher recurrence rate in subsequent years, which results in an overall modest prognostic significance. A useful aspect to having ER-alpha-positive cancer is in predicting response to hormonal therapy, both in the adjuvant setting and for advanced disease. Several therapies, including prevention and treatment, are available for ER-alpha positive cancers, although tamoxifen, a triphenylethilene derivative, is currently the most prevalent. Tamoxifen reduces proliferation of ER-alpha-positive cancer cells through an estradiol antagonist mechanism, although patients develop resistance to it. Moreover, the drug increases the risk for endometrial cancer.

ER-alpha is commonly depicted as a transcription factor which, once bound to the appropriate hormone (e.g., 1713-estradiol; E₂), regulates the expression of target genes. However, in view of discoveries made over the last decade, this simplistic concept must be reconsidered since the transcription of estrogen-regulated genes is actually a transient and cyclic mechanism involving successive recruitments and dissociations of a large number of corepressors and coactivators. These coregulators, which modulate ER-alpha activity to the same extent as cognate ligands, should not be viewed as “accessory proteins”, but rather as products of particular genes, historically called “master genes”, that orchestrate coherent and synchronized events. According to this view, the search for drugs able to specifically interfere with coregulator recruitment may open new therapeutic avenues for the treatment of ER-alpha-related diseases. In order to design such compounds, one must have a clear understanding of the molecular mechanisms underlying the formation of active ER-alpha oligomeric structures.

In this perspective, we previously described the regulatory function of the ER-alpha P₂₉₅-T₃₁₁ amino-acid sequence, which harbors a binding site for calmodulin (CaM) (Gallo et al., Mol Cell Endocrinol 2007; 268:37-49), a coregulator playing a role of major importance in the ER-alpha mechanism of action. The P₂₉₅-T₃₁₁ sequence, located between the D-(hinge) and E-(Ligand Binding Domain; LBD) domains, appeared to be involved in both the stability and the transcriptional activity of the receptor. This short sequence, actually situated in the AF-2a (autonomous activation function) domain, can be considered as a platform for various posttranslational modifications such as phosphorylation, acetylation, SUMOylation, monoubiquitination and methylation (see Gallo et al., Mol Cell Endocrinol 2008; 291:20-26). This motif also contains the third nuclear localization signal of the receptor, as well as a proteolysis site and a binding site for CaM, a coregulator which enhances both the transactivation and the stabilization of the receptor by impeding its E6-AP-(E6-Associated Protein) mediated polyubiquitination (Li et al., J Biol Chem 2006; 281:1978-85). Surprisingly, the ER-alpha-17p peptide was shown to elicit estrogenic responses in ER-alpha-expressing breast carcinoma cells (Gallo et al., Mol Cell Endocrinol 2007; 268:37-49; Gallo et al., Mol Cell Endocrinol 2007; 268:37-49; Gallo et al., J Steroid Biochem Mol Biol 2008; 109:138-149). The mechanism by which ER-alpha-17p operates is not established as yet, although their intracellular penetration seems to be required. In this regard, it should be stressed that ER-alpha, like other nuclear hormone receptors, is subject to a constant trafficking between various intracellular compartments and targets, especially in the absence of (anti)estrogenic stimulation. Note that in this context we do not know if a particular or all forms of ER-alpha (nuclear, cytoplasmic, membrane-associated, posttranslationally modified, etc.) are implicated in the mode of action of ER-alpha-17p. Recent investigations from our laboratory have revealed that ER-alpha-17p binds to purified recombinant human ER-alpha (Gallo et al., Letters in Drug Design & Discovery 2007; 4:346-355.), most likely disrupting intramolecular interactions suspected to confer upon the receptor an inactive conformation. The mechanism underlying these observations was however not elucidated, making it impossible to use it in e.g. anti-cancer drug design.

The present invention has investigated further the underlying mechanism and provides new and interesting possibilities for anti-cancer treatment based on these findings.

### SUMMARY OF THE INVENTION

The present invention is built around the surprising observation that an endogenous polypeptide breakdown product/peptide of the ER-alpha protein can be detected in the extracellular space of breast cancer cells when treated with estradiol (E₂). In addition, the inventors have established that treatment of ER-alpha-positive breast cancer cells with a synthetic peptide corresponding to this breakdown product/peptide increases their proliferation. Accordingly, sequestering said breakdown product/peptide using e.g. specific binding molecules to the endogenous polypeptide, significantly reduces the proliferation rate of said breast cancer cells in the presence of E₂.

The present invention thus generally provides new methods for diagnosing estrogen receptor positive (ER-alpha-positive) disorders. Possible disorders envisaged by the methods of the invention are ER-alpha-positive cancers such as breast cancer, endometrial cancer, cervical cancer or ovarian cancer, preferably breast cancer.

In a specific embodiment, the present invention provides methods for diagnosing ER-alpha-related disorders such as breast cancer, endometrial cancer, cervical cancer or ovarian cancer, preferably breast cancer in a subject comprising the steps of:

a) detecting in a sample of a subject under analysis, the concentration of an endogenous degradation product/peptide of the estrogen receptor-alpha (ER-alpha) protein consisting of any one of the sequences defined by SEQ ID NOs:2 to 29, or 32 to 225, wherein the fragment of SEQ ID NO:29, carries a methylated lysine at position K₃₀₃, and
b) comparing the obtained concentration of said peptide in the sample of the subject to a control concentration of said peptide in a healthy subject, wherein an elevated concentration of said peptide indicates the subject may be suffering from said ER-alpha-related disorder.

In a specific embodiment, the degradation product/peptide to be detected is a peptide defined by SEQ ID NO:2, (i.e. K₂₆₈RQRDDGEGRGEVGSAGDMRAANLWPSPLMIKRSKKNSLALSLT₃₁₁) of the full-length ER-alpha protein (gene: ERS1). In an even more preferred embodiment, the peptide detected is defined by SEQ ID NO:2 and comprises one or more of the following post-translational modifications: methylation of K₂₆₈, R₂₆₉, K₃₀₃, and/or oxidation of M₂₈₆, and/or phosphorylation of S₂₈₂.

In a further preferred embodiment, said endogenous degradation product/peptide of the ER-alpha is P₂₉₅LMIKRSKK₃₀₃NSLALSLT₃₁ carrying a methylated lysine at position 303 (SEQ ID NO:29).

In preferred embodiments, the method according to the invention is used for diagnosing ER-alpha-positive cancers selected from the group consisting of: breast cancer, endometrial cancer, cervical cancer and ovarian cancer, preferably breast cancer.

In preferred embodiments, the sample used in the method according to the invention is a sample of bodily fluid or tissue that is in direct contact or in close proximity to the tumour or cancer cells, i.e. samples comprising the secreted ER-alpha polypeptide of the invention in detectable quantities e.g. blood, acsites fluid, fluid surrounding tumour or cancer cells, etc. Most preferably, said sample is a serum sample of the subject.

The invention further provides a composition comprising an isolated endogenous ER-alpha peptide consisting of the amino acid sequence of SEQ ID NO:2, or a fragment thereof, preferably comprising the isolated endogenous ER-alpha peptide consisting of the amino acid sequence of SEQ ID NO:2; most preferably comprising one or more of the following post-translational modifications: methylation of K₂₆₈, R₂₆₉, K₃₀₃, and/or oxidation of M₂₈₆, and/or phosphorylation of S₂₈₂.

The composition can comprise the endogenous ER-alpha peptide consisting of the amino acid sequence of SEQ ID NO:2, or any fragment thereof that minimally comprises the 17 amino acid sequence PLMIKRSKKNSLALSLT, in which the K₃₀₃ residue can be methylated.

Alternatively, composition comprises any one of the following fragments of the endogenous ER-alpha polypeptide according to the present invention, consisting of the sequence:

### K₂₆₈RQRDDGEGRGEVGS₂₈₂AGDM₂₈₆RAANLWPSPLMIKRSKK₃₀₃NSLALSLT₃₁₁ (SEQ ID NO:2),

### R₂₆₉QRDDGEGRGEVGS₂₈₂AGDM₂₈₆RAANLWPSPLMIKRSKK₃₀₃NSLALSLT₃₁₁ (SEQ ID NO:3),

### Q₂₇₀RDDGEGRGEVGS₂₈₂AGDM₂₈₆RAANLWPSPLMIKRSKK₃₀₃NSLALSLT₃₁₁ (SEQ ID NO:4),

### R₂₇₁DDGEGRGEVGS₂₈₂AGDM₂₈₆RAANLWPSPLMIKRSKK₃₀₃NSLALSLT₃₁₁ (SEQ ID NO:5),

### D₂₇₂DGEGRGEVGS₂₈₂AGDM₂₈₆RAANLWPSPLMIKRSKK₃₀₃NSLALSLT₃₁₁ (SEQ ID NO:6),

### D₂₇₃GEGRGEVGS₂₈₂AGDM₂₈₆RAANLWPSPLMIKRSKK₃₀₃NSLALSLT₃₁₁ (SEQ ID NO:7),

### G₂₇₄EGRGEVGS₂₈₂AGDM₂₈₆RAANLWPSPLMIKRSKK₃₀₃NSLALSLT₃₁₁ (SEQ ID NO:8),

### E₂₇₅GRGEVGS₂₈₂AGDM₂₈₆RAANLWPSPLMIKRSKK₃₀₃NSLALSLT₃₁₁ (SEQ ID NO:9),

### G₂₇₆RGEVGS₂₈₂AGDM₂₈₆RAANLWPSPLMIKRSKK₃₀₃NSLALSLT₃₁₁ (SEQ ID NO:10),

### R₂₇₇GEVGS₂₈₂AGDM₂₈₆RAANLWPSPLMIKRSKK₃₀₃NSLALSLT₃₁₁ (SEQ ID NO:11),

### G₂₇₈EVGS₂₈₂AGDM₂₈₆RAANLWPSPLMIKRSKK₃₀₃NSLALSLT₃₁₁ (SEQ ID NO:12),

### E₂₇₉VGS₂₈₂AGDM₂₈₆RAANLWPSPLMIKRSKK₃₀₃NSLALSLT₃₁₁ (SEQ ID NO:13),

### V₂₈₀GS₂₈₂AGDM₂₈₆RAANLWPSPLMIKRSKK₃₀₃NSLALSLT₃₁₁ (SEQ ID NO:14),

### G₂₈₁S₂₈₂AGDM₂₈₆RAANLWPSPLMIKRSKK₃₀₃NSLALSLT₃₁₁ (SEQ ID NO:15),

### S₂₈₂AGDM₂₈₆RAANLWPSPLMIKRSKK₃₀₃NSLALSLT₃₁₁ (SEQ ID NO:16),

### A₂₈₃GDM₂₈₆RAANLWPSPLMIKRSKK₃₀₃NSLALSLT₃₁₁ (SEQ ID NO:17),

### G₂₈₄DM₂₈₆RAANLWPSPLMIKRSKK₃₀₃NSLALSLT₃₁₁ (SEQ ID NO:18),

### D₂₈₅M₂₈₆RAANLWPSPLMIKRSKK₃₀₃NSLALSLT₃₁₁ (SEQ ID NO:19),

### M₂₈₆RAANLWPSPLMIKRSKK₃₀₃NSLALSLT₃₁₁ (SEQ ID NO:20),

### R₂₈₇AANLWPSPLMIKRSKK₃₀₃NSLALSLT₃₁₁ (SEQ ID NO:21),

### A₂₈₈ANLWPSPLMIKRSKK₃₀₃NSLALSLT₃₁₁ (SEQ ID NO:22),

### A₂₈₉NLWPSPLMIKRSKK₃₀₃NSLALSLT₃₁₁ (SEQ ID NO:23),

### N₂₉₀LWPSPLMIKRSKK₃₀₃NSLALSLT₃₁₁ (SEQ ID NO:24),

### L₂₉₁WPSPLMIKRSKK₃₀₃NSLALSLT₃₁₁ (SEQ ID NO:25),

### W₂₉₂PSPLMIKRSKK₃₀₃NSLALSLT₃₁₁ (SEQ ID NO:26),

### P₂₉₃SPLMIKRSKK₃₀₃NSLALSLT₃₁₁ (SEQ ID NO:27),

### S₂₉₄PLMIKRSKK₃₀₃NSLALSLT₃₁₁ (SEQ ID NO:28),

### P₂₉₅LMIKRSKK₃₀₃NSLALSLT₃₁₁ (SEQ ID NO:29), or

any one of the sequences defined by SEQ ID Nos 32 to 225,
wherein in any one of the sequences listed above, one or more of the following post translational modifications can be present: methylation of K₂₆₈, R₂₆₉, K₃₀₃, and/or oxidation of M₂₈₆, and/or phosphorylation of S₂₈₂. The subscripts used in said sequences refer to the positions of the corresponding residues in the full-length ER-alpha amino acid sequence.

In a specific embodiment of said composition, the degradation product/peptide to be detected is a peptide defined by SEQ ID NO:2, (i.e. K₂₆₈RQRDDGEGRGEVGSAGDMRAANLWPSPLMIKRSKKNSLALSLT₃₁₁) of the full-length ER-alpha protein (gene: ERS1). In an even more preferred embodiment, the peptide detected is defined by SEQ ID NO:2 and comprises one or more of the following post-translational modifications: methylation of K₂₆₈, R₂₆₉, K₃₀₃, and/or oxidation of M₂₈₆, and/or phosphorylation of S₂₈₂.

In a further preferred embodiment of said composition, said endogenous degradation product/peptide of the ER-alpha is P₂₉₅LMIKRSKK₃₀₃NSLALSLT₃₁ carrying a methylated lysine at position 303 (SEQ ID NO:29).

In a further preferred embodiment, the composition comprises an antigenic fragment of said endogenous ER-alpha degradation product/peptide, as defined by any one of SEQ ID Nos 32 to 225.

The composition of the present invention can be linked to a carrier molecule such as bovine serum albumine (BSA) or keyhole limpit hemocyanine (KLH), or can be comprised within a lipid composition such as, a lipid particle, a nanocapsule, a liposome, or lipid vesicle. Preferably, said composition comprises a pharmaceutical excipient.

The composition according to the present invention can be used in vaccination against ER-alpha-positive cancers. The present invention thus provides for the use of the composition according to the invention for use as a vaccine and/or for use in vaccination. In a preferred embodiment, said vaccination is against human breast cancer.

The composition in accordance with the invention can be for use in preparing a vaccine for preventing or treating ER-alpha-positive cancers or for use in preparing a cancer inhibitor that specifically binds to the estrogen receptor polypeptide.

The invention further provides for the use of a composition in accordance with the invention in the manufacture of a medicament for use in treating ER-alpha-positive cancers, preferably breast cancer.

Instead of a protein or polypeptide as such, the present invention also provides for nucleotides encoding any one of the ER-alpha degradation product polypeptides or fragments as defined herein.

The invention further provides for the use of such a nucleic acid according to the invention for use in DNA-vaccination; wherein said nucleic acid is transferred into the host cell, where it expresses the polypeptide of the invention (e.g. of SEQ ID NO:2), to which an immune response is to be elicited e.g. in order to reduce cancer growth. Delivery of said nucleic acid can be done by direct administration of the DNA molecule, or can be done through a bacterial or viral expression system known in the art of gene therapy.

The invention thus provides for an isolated nucleic acid that encodes any one of the peptides containing the sequence defined by SEQ ID Nos 2 to 29, or 32 to 225. In a preferred embodiment, said nucleic acid is defined by SEQ ID NO:1 and encodes the polypeptide as defined in SEQ-ID NO:2.

The invention thus provides for the nucleic acids as defined herein for use in preparing a medicament or vaccine for treating ER-alpha-positive cancers or for the use of said nucleic acid in the manufacture of a medicament or vaccine for use in treating ER-alpha-positive cancers. In a preferred embodiment, said nucleic acid is in the form of RNA, mRNA, DNA, or cDNA.

In addition, the invention provides for a recombinant vector that expresses a (poly)peptide as defined herein. Such a recombinant vector can also be used for gene-therapy, whereby said nucleic acid as defined herein is transferred to the host and made to express the (poly)peptide it is encoding into said host, thereby eliciting an immune response to said polypeptide in the host. Said recombinant vector can be for use in preparing a medicament or vaccine for treating cancer or can be used in the manufacture of a medicament or vaccine for use in treating cancer. Viral delivery of RNA or DNA encoding the polypeptide according to the invention for vaccination purposes is also envisaged using known methodologies.

Furthermore, a recombinant host cell expressing the nucleic acid as defined herein, polypeptide as defined herein or the recombinant vector as defined herein is envisaged by the present invention, as well as its use in preparing a medicament or vaccine for treating cancer.

Preferably, said host cell is a mammalian cell, e.g. a human cell, a bacterial cell, e.g. an E. coli, Salmonella, or Pseudomonas cell.

The invention further provides for a method of reducing estrogen receptor activity in a cell, comprising providing to said cell an effective inhibitory amount of an inhibitor composition as defined herein.

In a preferred embodiment, said cell is comprised within an animal, and said composition is administered to said animal, preferably, said cell is an animal cell, more preferably said cell is comprised within an animal that has cancer, preferably breast cancer.

The invention further provides for a method of reducing proliferation of a cancer cell, comprising providing to said cancer cell a therapeutically effective amount of an inhibitor of the ER-alpha receptor as defined herein.

In addition, the invention provides for a method of vaccinating a subject comprising the steps of providing to said subject a polypeptide as defined herein, a nucleic acid as defined herein, a recombinant vector as defined herein or a host cell as defined herein.

The invention furthermore provides for a method of treating ER-alpha-positive cancers in a subject, comprising the steps of:

(a) identifying a subject having increased estrogen receptor activity; and
(b) administering to said subject a therapeutically-effective amount of the inhibitor of ER-alpha, ER-alpha-vaccine or composition according to the present invention.

Preferably, said composition is formulated in a pharmaceutical excipient for administration intravenously, parenterally, orally, topically, or as an inhalant, aerosol or spray. Preferably, said subject is a human.

In an alternative embodiment, the method of treating ER-alpha-positive cancers in an animal further comprises administering at least a second anticancer agent to said animal, such as cyclophosphamide, methotrexate, fluorouracil, adriamycin, tamoxifen, doxorubicin, etoposide, verapamil, podophyllotoxin, and an analog or salt thereof.

The invention furthermore provides for binding molecules that specifically bind to the ER-alpha degradation product/peptide as defined herein, preferably to the polypeptide consisting of the amino acid sequence of SEQ ID NO:2. Preferably, said binding molecule specifically binds to the polypeptide as defined in SEQ ID NO:2, comprising one or more of the following post-translational modifications: methylation of K₂₆₈, R₂₆₉, K₃₀₃, and/or oxidation of M₂₈₆, and/or phosphorylation of S₂₈₂. In a further embodiment, said antibody binds to a fragment of the endogenous ER-alpha degradation product/peptide as defined herein, i.e. to any one of the (poly)peptides consisting of the sequence defined by SEQ ID Nos 1 to 29, or 32 to 225.

Examples of binding molecules envisaged hereby are antibodies, monoclonal- or polyclonal antibodies, nanobodies, affybodies, antibody fragments, aptamers, photoaptamers, oligonucleotides, lipocalins, specifically interacting small molecules, Molecular Imprinting Polymers (MIPs), DARPins, ankyrins, specifically interacting proteins, peptidomimetics, biomimetics or peptides, and other molecules that specifically bind to said polypeptide. Both monoclonal, polyclonal or single chain antibodies or fragments thereof that bind one of the biomarkers of the present invention are useful in the methods and kits of the present invention.

Such binding molecules can also act as inhibitors of the ER-alpha function.

The invention thus also provides a pharmaceutical composition comprising an inhibitor of ER-alpha for treating cancer or for use in the manufacturing of a medicament for treating cancer. Preferably, said inhibitor is a binding molecule as defined herein, e.g. specifically binding to the endogenous ER-alpha degradation product/peptide of the invention as defined by SEQ ID NO:2, optionally specifically binding to the polypeptide of the invention as defined by SEQ ID NO:2 comprising one or more of the following post-translational modifications: methylation of K₂₆₈, R₂₆₉, K₃₀₃, and/or oxidation of M₂₈₆, and/or phosphorylation of S₂₈₂.

In a preferred embodiment, said binding molecule is an antibody or fragment thereof.

In a preferred embodiment, said binding molecule binds specifically to a fragment of the endogenous ER-alpha degradation product/peptide as defined herein, i.e. to any one of the (poly)peptides consisting of the sequence defined by SEQ ID Nos 1 to 29, or 32 to 225.

The antibody according to the present invention can alternatively be attached to a detectable label.

The inhibitors or binding molecules as indicated above can also be used independently, i.e. not in a pharmaceutical composition, for example for the detection of said polypeptide of the invention. The invention thus provides for inhibitors or binding molecules of the ER-alpha receptor protein as such, defined by their ability to bind specifically to the endogenous ER-alpha degradation product/peptide as defined herein, preferably defined by SEQ ID NO:2, optionally specifically binding to the polypeptide of the invention as defined by SEQ ID NO:2 comprising one or more of the following post-translational modifications: methylation of K₂₆₈, R₂₆₉, K₃₀₃, and/or oxidation of M₂₈₆, and/or phosphorylation of S₂₈₂.

In a preferred embodiment, said binding molecule is an antibody or fragment thereof.

In a preferred embodiment, said binding molecule binds specifically to a fragment of the endogenous ER-alpha degradation product/peptide as defined herein, i.e. to any one of the (poly)peptides consisting of the sequence defined by SEQ ID Nos 1 to 29, or 32 to 225.

In particular, the invention thus provides the following points:

1. A method for diagnosing estrogen receptor alpha positive cancer in a subject comprising the steps of:
a) detecting in a sample of a subject under analysis, the concentration of an endogenous degradation peptide of the estrogen receptor-alpha (ER-alpha) consisting of any one of the sequences defined by SEQ ID NOs:2 to 29, or 32 to 225, wherein the fragment of SEQ ID NO:29, carries a methylated lysine at position K₃₀₃, and
b) comparing the obtained concentration of said peptide in the sample of the subject to a control concentration of said peptide in a healthy subject, wherein an elevated concentration of said peptide indicates the subject may be suffering of an ER-alpha-positive cancer.
2. The method according to point 1, wherein said endogenous degradation peptide of the ER-alpha is P₂₉₅LMIKRSKK₃₀₃NSLALSLT₃₁ carrying a methylated lysine at position 303 (SEQ ID NO:29).
3. The method according to point 1 or 2, wherein said endogenous degradation peptide consists of the sequence of SEQ ID NO:2.
4. The method according to any one of points 1 to 3, wherein said endogenous degradation peptide carriers one or more of the following post-translational modifications: methylation of K₂₆₈, R₂₆₉, K₃₀₃, and/or oxidation of M₂₈₆, and/or phosphorylation of S₂₈₂.
5. The method according to any one of points 1 to 4, wherein said ER-alpha-positive cancer is preferably selected from the group consisting of: breast cancer, endometrial cancer, cervical cancer and ovarian cancer, most preferably human breast cancer.
6. The method according to any one of points 1 to 5, wherein the sample is preferably selected from the consisting of: whole blood, plasma, serum, nipple aspirate, ductal lavage, tumour exudates, tumour cavity fluid, pleural effusion, acsites fluid, fluid surrounding tumour or cancer cells, lymph, any other bodily fluid in close contact with the tumour or cancer.
7. An isolated estrogen receptor-alpha (ER-alpha) polypeptide consisting of any one of the sequences defined by SEQ ID NOs:2 to 29, or 32 to 225, wherein the fragment of SEQ ID NO:29, carries a methylated lysine at position K₃₀₃,
8. The isolated polypeptide according to point 7, carrying one or more of the following post-translational modifications: methylation of K₂₆₈, R₂₆₉, K₃₀₃, and/or oxidation of M₂₈₆, and/or phosphorylation of S₂₈₂.
9. A composition comprising the isolated ER-alpha polypeptide according to point 7 or 8, wherein said peptide is linked to a carrier molecule such as bovine serum albumine or keyhole limpet hemocyanin, or comprised within a lipid composition such as, a lipid particle, a nanocapsule, a liposome, or lipid vesicle, optionally further comprising a pharmaceutical excipient.
10. The composition according to point 9, additionally comprising an adjuvant.
11. The polypeptide according to point 7 or 8 or the composition according to point 9 or 10, for use in treatment of, or vaccination against ER-alpha-positive cancers, preferably breast cancer, endometrial cancer, cervical cancer and ovarian cancer, most preferably human breast cancer.
12. A nucleic acid molecule encoding the polypeptide according to point 7.
13. The nucleic acid according to point 12, consisting of a polynucleotide sequence having at least 90% identity to SEQ ID NO.1, more preferably consisting of the polynucleotide sequence of SEQ ID NO:1.
14. The nucleic acid molecule according to point 12 or 13, for use in treatment of, or vaccination against ER-alpha-positive cancers, preferably breast cancer, endometrial cancer, cervical cancer and ovarian cancer, most preferably human breast cancer.
15. A recombinant vector that expresses a peptide according to point 7, or comprises the nucleic acid molecule according to point 12 or 13.
16. The recombinant vector according to point 15, for treatment of, or vaccinating against ER-alpha-positive cancers, preferably breast cancer, endometrial cancer, cervical cancer and ovarian cancer, most preferably human breast cancer.
17. A host cell comprising a polypeptide according to point 7 or 8, the composition according to point 9 or 10, the nucleic acid molecule according to point 12 or 13, or the recombinant vector according to point 14, wherein said cell preferably is a mammalian cell, e.g. a human cell, a yeast cell, a bacterial cell, e.g. an E. coli, Salmonella, or Pseudomonas cell.
18. The host cell according to point 17, for treatment of, or vaccinating against ER-alpha-related diseases, preferably breast cancer, endometrial cancer, cervical cancer and ovarian cancer, most preferably human breast cancer.
19. A purified binding molecule that specifically binds to the polypeptide according to point 7 or 8, preferably antibodies, monoclonal- or polyclonal antibodies, nanobodies, affybodies, antibody fragments, aptamers, photoaptamers, oligonucleotides, lipocalins, specifically interacting small molecules, Molecular Imprinting Polymers (MIPs), DARPins, ankyrins, specifically interacting proteins, peptidomimetics, biomimetics or peptides, and other molecules that specifically bind to said polypeptide.
20. An immunodetection kit comprising:
a) a binding molecule according to point 19,
b) a reference value of the amount of corresponding peptide to which said binding molecule specifically binds, in a healthy subject and
c) instructions to compare the amounts of said peptide in a sample of the subject under investigation and in a sample of a healthy subject in order to conclude whether said subject has ER-alpha positive cancer or not.
21. The use of the polypeptide according to point 7 or 8, as a biomarker for diagnosing, predicting, prognosticating and/or monitoring an ER-alpha positive cancer, preferably breast cancer, endometrial cancer, cervical cancer and ovarian cancer, most preferably human breast cancer.
22. A method of treating a subject having ER-alpha-positive cancer, comprising administering to said subject a therapeutically effective amount of the peptide according to point 7 or 8, or the composition according to point 9 or 10.
23. A method for vaccinating a subject against the occurrence of ER-alpha-positive cancer, comprising administering to said subject a therapeutically effective amount of the peptide according to point 7 or 8, or the composition according to point 9 or 10.
24. A method of treating ER-alpha-positive cancers, preferably breast cancer, endometrial cancer, cervical cancer and ovarian cancer, most preferably human breast cancer, comprising the steps of administering the polypeptide according to point 7 or 8, the composition according to point 9 or 10, the nucleic acid molecule according to point 12 or 13, the recombinant vector according to point 15, or the host cell according to point 17, to a subject in need thereof, optionally combined with other therapeutic agents that can be beneficial or synergistic to the treatment of said subject.
25. A method of vaccinating a subject in need thereof against developing ER-alpha-positive cancers, preferably breast cancer, endometrial cancer, cervical cancer and ovarian cancer, most preferably human breast cancer, comprising the steps of administering the polypeptide according to point 7 or 8 or the composition according to point 9 or 10, the nucleic acid molecule according to point 12 or 13, the recombinant vector according to point 15, or the host cell according to point 17, to said subject, optionally combined with other therapeutic agents or with adjuvants that can be beneficial or synergistic to the vaccination of said subject.
26. The use of the polypeptide according to point 7 or 8 or the composition according to point 9 or 10, the nucleic acid molecule according to point 12 or 13, the recombinant vector according to point 15, or the host cell according to point 17, for the manufacturing of a medicament for treating ER-alpha-positive cancers, preferably breast cancer, endometrial cancer, cervical cancer and ovarian cancer, most preferably human breast cancer in a subject.
27. The use of the polypeptide according to point 7 or 8 or the composition according to point 9 or 10, the nucleic acid molecule according to point 12 or 13, the recombinant vector according to point 15, or the host cell according to point 17, for the manufacturing of a medicament for vaccinating a subject against developing ER-alpha-positive cancers, preferably breast cancer, endometrial cancer, cervical cancer and ovarian cancer, most preferably human breast cancer.

### BRIEF DESCRIPTION OF THE DRAWINGS

FIG. 1: MS/MS Spectrum of an endogenous peptide extracted form conditioned medium of E₂-stimulated MCF-7 cells. This peptide encompasses the amino acid sequence K₂₆₈-T₃₁₁ and, thus, the sequence of the ER-alpha-17p (precursor charged 6×; m/z=823,26).

FIG. 2: Sequence and post-translational modifications of the detected peptide (SEQ ID NO:2) and the corresponding nucleic acid sequence (SEQ ID NO:1) encoding said peptide.

FIG. 3: Immunoprecipitation of the endogenous peptide. The peptide was extracted from conditioned medium of MCF-7 cells treated or not (control) with estradiol (10⁻⁸M), diethylstibestrol (10⁻⁸M), 4-OHTamoxifen (10⁻⁷M) or Fulvestrant (10⁻⁷M).

FIG. 4: Cell growth in the presence or absence of a synthetic peptide corresponding to the ER-alpha breakdown product/peptide (SEQ ID NO:2). MCF-7 cells were incubated with or without (control) ER-alpha-17p or SEQ ID NO:2 peptide both at 10⁻⁵M for 72 h. Growth was assessed by crystal violet staining.

FIG. 5: Cell growth in the presence or absence of E₂ at 10⁻¹¹ and10⁻¹²M with or without an antibody (dilution 1/100) raised against the C-terminal part of the ER-alpha breakdown product/peptide (SEQ ID NO:2) for 72 h. Growth was assessed by crystal violet staining.

FIG. 6: Transcriptional activity of ER-alpha. MVLN cells (MCF-7 stably transfected with an ERE-driven luciferase reporter gene; Pons et al., Biotechniques 1990; 9:450-459) in the presence or absence (control) of E₂ at 10⁻¹⁰ M or ER-alpha-17p or SEQ ID NO:2 peptide both at 10⁻⁵M for 6 h in serum free condition.

FIG. 7: cDNA coding sequence of ER-alpha isoform 1 (Genbank Acc. No. NM_{—}000125 (SEQ ID NO:3)). The bold sequence corresponds to SEQ ID NO:1, encoding the peptide of SEQ ID NO:2.

FIG. 8: Amino acid sequence of ER-alpha isoform 1 (Genbank Acc. No. NP_{—}000116 (SEQ ID NO: 4)), indicating the identified polypeptide according to the invention (SEQ ID NO:2).

### DETAILED DESCRIPTION OF THE PREFERRED EMBODIMENTS

As used herein, the terms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

All documents cited in the present specification are hereby incorporated by reference in their entirety.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

The terms “predicting” or “prediction”, “diagnosing” or “diagnosis” and “prognosticating” or “prognosis” are commonplace and well-understood in medical and clinical practice. By way of example only and without limitation, “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population.

The terms “diagnosing” or “diagnosis” generally refer to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosticating” or “prognosis” generally refer to an anticipation on the progression of a disease or condition and the prospect (e.g., the probability, duration, and/or extent) of recovery.

The term “subject” or “patient” as used herein typically denotes humans, but may also encompass reference to non-human animals, preferably warm-blooded animals, more preferably mammals, such as, e.g., non-human primates, rodents, canines, felines, equines, ovines, porcines, and the like.

The terms “sample” or “biological sample” as used herein include any biological specimen obtained from a subject. Samples may include, without limitation, whole blood, plasma, serum, red blood cells, white blood cells (e.g., peripheral blood mononuclear cells), saliva, urine, stool (i.e., faeces), tears, sweat, sebum, nipple aspirate, ductal lavage, tumour exudates, tumour cavity fluid, pleural effusion, synovial fluid, cerebrospinal fluid, lymph, fine needle aspirate, amniotic fluid, any other bodily fluid, cell lysates, cellular secretion products, inflammation fluid, semen and vaginal secretions. Preferred samples may include ones comprising the secreted ER-alpha polypeptide of the invention in detectable quantities e.g. blood, acsites fluid, fluid surrounding tumour or cancer cells, etc. In preferred embodiments, the sample may be whole blood or a fractional component thereof such as, e.g., plasma, serum, or a cell pellet. Preferably the sample is readily obtainable by minimally invasive methods. Samples may alternatively also include tissue samples and biopsies, tissue homogenates and the like.

The terms “quantity”, “amount” and “level” are synonymous and generally well-understood in the art. The terms as used herein may particularly refer to an absolute quantification of a molecule or an analyte in a sample, or to a relative quantification of a molecule or analyte in a sample, i.e., relative to another value such as relative to a reference value as taught herein. An absolute quantity of a molecule or analyte in a sample may be advantageously expressed as weight or as molar amount, or more commonly as a concentration, e.g., weight per volume or mol per volume. A relative quantity of a molecule or analyte in a sample may be advantageously expressed as an increase or decrease or as a fold-increase or fold-decrease relative to said another value, such as relative to a reference value as taught herein. Performing a relative comparison between first and second parameters (e.g., first and second quantities) may but need not require to first determine the absolute values of said first and second parameters. For example, a measurement method can produce quantifiable readouts (such as, e.g., signal intensities) for said first and second parameters, wherein said readouts are a function of the value of said parameters, and wherein said readouts can be directly compared to produce a relative value for the first parameter vs. the second parameter, without the actual need to first convert the readouts to absolute values of the respective parameters.

Also provided are binding molecule capable of specifically binding to the ER-alpha polypeptide as taught herein preferably defined by SEQ ID NO:2 or to its specific forms comprising one or more of the following post-translational modifications: methylation of K₂₆₈, R₂₆₉, K₃₀₃, and/or oxidation of M₂₈₆, and/or phosphorylation of S₂₈₂. Such binding molecules may include inter alia an antibody, aptamer, photoaptamer, protein, peptide, peptidomimetic or a small molecule.

The term “specifically bind” as used throughout this specification means that an agent (denoted herein also as “specific binding molecule” or “specific binding molecule”) binds to one or more desired molecules or analytes, such as to one or more proteins, polypeptides or peptides of interest or fragments thereof substantially to the exclusion of other molecules which are random or unrelated, and optionally substantially to the exclusion of other molecules that are structurally related.

The term “specifically bind” does not necessarily require that a molecule binds exclusively to its intended target(s). For example, a molecule may be said to specifically bind to the target polypeptide(s), peptide(s) and/or fragment(s) thereof of the invention or their post-translationally modified forms, if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold or more greater, than its affinity for a non-target polypeptide or molecule.

Preferably, the binding molecule may bind to its intended target(s) with affinity constant (K_{A}) of such binding K_{A}≧1×10⁶ M⁻¹, more preferably K_{A}≧1×10⁷ M⁻¹, yet more preferably K_{A}≧1×10⁸ M⁻¹, even more preferably K_{A}≧1×10⁹ M⁻¹, and still more preferably K_{A}≧1×10¹⁰ M⁻¹ or K_{A}≧1×10¹¹ M⁻¹, wherein K_{A}=[SBM_T]/[SBM][T], SBA denotes the specific-binding molecule, T denotes the intended target. Determination of K_{A} can be carried out by methods known in the art, such as for example, using equilibrium dialysis and Scatchard plot analysis.

Specific-binding molecules as used throughout this specification may include inter alia an antibody, aptamer, photoaptamer, protein, peptide, peptidomimetic or a small molecule.

As used herein, the term “antibody” is used in its broadest sense and generally refers to any immunologic binding molecule that is specifically binding to a target molecule such as the ER-alpha polypeptide as taught herein or one or more of its post-translationally modified forms as defined herein. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro or in vivo.

In an embodiment, an antibody may be any of IgA, IgD, IgE, IgG and IgM classes, and preferably IgG class antibody. For therapeutic purposes, IgG antibodies are preferred, especially of the IgG2 or IgG4 type, which induce a lower autoimmune response in the subject.

In an embodiment, the antibody may be a polyclonal antibody, e.g., an antiserum or immunoglobulins purified there from (e.g., affinity-purified).

In another preferred embodiment, the antibody may be a monoclonal antibody or a mixture of monoclonal antibodies. Monoclonal antibodies can target a particular antigen or a particular epitope within an antigen with greater selectivity and reproducibility.

By means of example and not limitation, monoclonal antibodies may be made by the hybridoma method first described by Kohler et al. 1975 (Nature 256: 495), or may be made by recombinant DNA methods (e.g., as in U.S. Pat. No. 4,816,567). Monoclonal antibodies may also be isolated from phage antibody libraries using techniques as described by Clackson et al. 1991 (Nature 352: 624-628) and Marks et al. 1991 (J Mol Biol 222: 581-597), for example.

In further embodiments, the antibody binding molecules may be antibody fragments. “Antibody fragments” comprise a portion of an intact antibody, comprising the antigen-binding or variable region thereof. Examples of antibody fragments include Fab, Fab′, F(ab′)2, Fv and scFv fragments; diabodies; linear antibodies; single-chain antibody molecules; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse.

A skilled person will understand that an antibody can include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

The term “aptamer” refers to single-stranded or double-stranded oligo-DNA, oligo-RNA or oligo-DNA/RNA or any analogue thereof, that can specifically bind to a target molecule such as the ER-alpha polypeptide as taught herein or one or more of its post-translationally modified forms as defined herein. Advantageously, aptamers can display fairly high specificity and affinity (e.g., K_{A} in the order 1×10⁹ M⁻¹) for their targets. Aptamer production is described inter alia in U.S. Pat. No. 5,270,163; Ellington & Szostak 1990 (Nature 346: 818-822); Tuerk & Gold 1990 (Science 249: 505-510); or “The Aptamer Handbook: Functional Oligonucleotides and Their Applications”, by Klussmann, ed., Wiley-VCH 2006, ISBN 3527310592, incorporated by reference herein.

The term “photoaptamer” refers to an aptamer that contains one or more photoreactive functional groups that can covalently bind to or crosslink with a target molecule.

The term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol 13: 132-134).

The term “small molecule” refers to compounds, preferably organic compounds, with a size comparable to those organic molecules generally used in pharmaceuticals. The term excludes biological macromolecules (e.g., proteins, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. Preferred small molecules are those that specifically bind to a target molecule such as the ER-alpha polypeptide as taught herein or one or more of its post-translationally modified forms as defined herein.

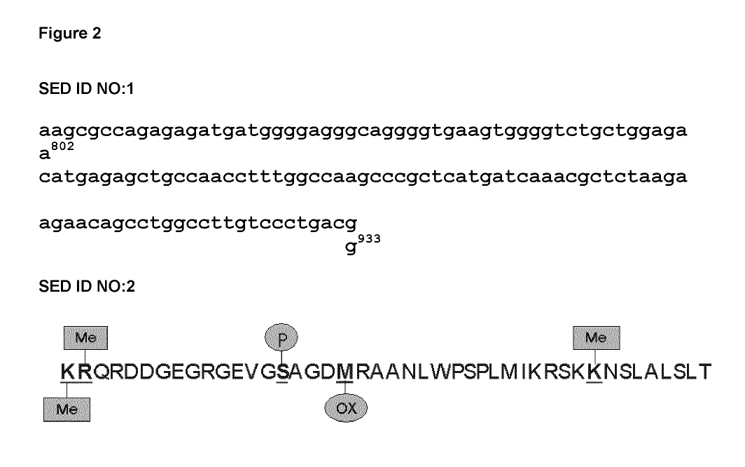

Also provided are methods for preparing specific antibodies directed to the peptide of the invention (SEQ ID NO:2), by immunising animals, e.g., non-human animals such as laboratory or farm, animals using (i.e., using as the immunising antigen) the ER-alpha polypeptide as taught herein (e.g. SEQ ID NO:2) or one or more of its post-translationally modified forms as defined herein (cf. FIG. 2), optionally attached to a presenting carrier. Alternatively DNA or RNA immunisation can be applied using the nucleotide defined herein (e.g. SEQ ID NO:1). Immunisation and preparation of antibody reagents from immune sera is well-known per se. The animals to be immunised may include any animal species, preferably warm-blooded species, more preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel, llama or horse. The term “presenting carrier” or “carrier” generally denotes an immunogenic molecule which, when bound to a second molecule, augments immune responses to the latter, usually through the provision of additional T cell epitopes. The presenting carrier may be a (poly)peptidic structure or a non-peptidic structure, such as inter alia glycans, polyethylene glycols, peptide mimetics, synthetic polymers, etc. Exemplary non-limiting carriers include human Hepatitis B virus core protein, multiple C3d domains, tetanus toxin fragment C or yeast Ty particles.

Immune sera obtained or obtainable by immunisation as taught herein may be particularly useful for generating antibody reagents that specifically bind to the ER-alpha polypeptide as taught herein or one or more of its post-translationally modified forms as defined herein.

The invention also teaches a method for selecting specific-binding molecules which bind specifically to the ER-alpha polypeptide as taught herein or one or more of its post-translationally modified forms as defined herein. Conveniently, such methods may be based on subtracting or removing binding molecules which cross-react or cross-bind the non-desired molecules. Such subtraction may be readily performed as known in the art by a variety of affinity separation methods, such as affinity chromatography, affinity solid phase extraction, affinity magnetic extraction, etc.

Any existing, available or conventional separation, detection and quantification methods can be used herein to measure the presence or absence (e.g., readout being present vs. absent; or detectable amount vs. undetectable amount) and/or quantity (e.g., readout being an absolute or relative quantity, such as, for example, absolute or relative concentration) of the ER-alpha polypeptide as taught herein or one or more of its post-translationally modified forms as defined herein.

For example, such methods may include immunoassay methods, mass spectrometry analysis methods, or chromatography methods, or combinations thereof.

The term “immunoassay” generally refers to methods known as such for detecting one or more molecules or analytes of interest in a sample, wherein specificity of an immunoassay for the molecule(s) or analyte(s) of interest is conferred by specific binding between a specific-binding molecule, commonly an antibody, and the molecule(s) or analyte(s) of interest.

Immunoassay technologies include without limitation direct ELISA (enzyme-linked immunosorbent assay), indirect ELISA, sandwich ELISA, competitive ELISA, multiplex ELISA, radioimmunoassay (RIA), ELISPOT technologies, FPIA (Fluorescence Polarization Immunoassay) and other similar techniques known in the art. Principles of these immunoassay methods are known in the art, for example John R. Crowther, “The ELISA Guidebook”, 1st ed., Humana Press 2000, ISBN 0896037282.

By means of further explanation and not limitation, direct ELISA employs a labelled primary antibody to bind to and thereby quantify target antigen in a sample immobilised on a solid support such as a microwell plate. Indirect ELISA uses a non-labelled primary antibody which binds to the target antigen and a secondary labelled antibody that recognises and allows to quantify the antigen-bound primary antibody. In sandwich ELISA the target antigen is captured from a sample using an immobilised ‘capture’ antibody which binds to one antigenic site within the antigen, and subsequent to removal of non-bound analytes the so-captured antigen is detected using a ‘detection’ antibody which binds to another antigenic site within said antigen, where the detection antibody may be directly labelled or indirectly detectable as above. Competitive ELISA uses a labelled ‘competitor’ that may either be the primary antibody or the target antigen. In an example, non-labelled immobilised primary antibody is incubated with a sample, this reaction is allowed to reach equilibrium, and then labelled target antigen is added. The latter will bind to the primary antibody wherever its binding sites are not yet occupied by non-labelled target antigen from the sample. Thus, the detected amount of bound labelled antigen inversely correlates with the amount of non-labelled antigen in the sample. Multiplex ELISA allows simultaneous detection of two or more analytes within a single compartment (e.g., microplate well) usually at a plurality of array addresses (see, for example, Nielsen & Geierstanger 2004. J Immunol Methods 290: 107-20 and Ling et al. 2007. Expert Rev Mol Diagn 7: 87-98 for further guidance). As appreciated, labelling in ELISA technologies is usually by enzyme (such as, e.g., horse-radish peroxidase) conjugation and the end-point is typically colorimetric, chemiluminescent or fluorescent.

Radioimmunoassay (RIA) is a competition-based technique and involves mixing known quantities of radioactively-labelled (e.g., ¹²⁵I- or ¹³¹I-labelled) target antigen with antibody to said antigen, then adding non-labelled or ‘cold’ antigen from a sample and measuring the amount of labelled antigen displaced (see, e.g., “An Introduction to Radioimmunoassay and Related Techniques”, by Chard T, ed., Elsevier Science 1995, ISBN 0444821198 for guidance).

Further, mass spectrometry methods are suitable for measuring biomarkers.

Generally, any mass spectrometric (MS) techniques that can obtain precise information on the mass of peptides, and preferably also on fragmentation and/or (partial) amino acid sequence of selected peptides (e.g., in tandem mass spectrometry, MS/MS; or in post source decay, TOF MS), are useful herein. Suitable peptide MS and MS/MS techniques and systems are well-known per se (see, e.g., Methods in Molecular Biology, vol. 146: “Mass Spectrometry of Proteins and Peptides”, by Chapman, ed., Humana Press 2000, ISBN 089603609x; Biemann 1990. Methods Enzymol 193: 455-79; or Methods in Enzymology, vol. 402: “Biological Mass Spectrometry”, by Burlingame, ed., Academic Press 2005, ISBN 9780121828073) and may be used herein.

MS arrangements, instruments and systems suitable for biomarker peptide analysis may include, without limitation, matrix-assisted laser desorption/ionisation time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)ⁿ (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)ⁿ; atmospheric pressure photoionization mass spectrometry (APPI-MS); APPI-MS/MS; and APPI-(MS)ⁿ. Peptide ion fragmentation in tandem MS (MS/MS) arrangements may be achieved using manners established in the art, such as, e.g., collision induced dissociation (CID).

In an embodiment, detection and quantification of biomarkers by mass spectrometry may involve multiple reaction monitoring (MRM), such as described among others by Kuhn et al. 2004 (Proteomics 4: 1175-86).

In an embodiment, MS peptide analysis methods may be advantageously combined with upstream peptide or protein separation or fractionation methods, such as for example with the chromatographic and other methods described herein below.

Chromatography can also be used for measuring biomarkers. As used herein, the term “chromatography” encompasses methods for separating chemical substances, referred to as such and vastly available in the art. In a preferred approach, chromatography refers to a process in which a mixture of chemical substances (analytes) carried by a moving stream of liquid or gas (“mobile phase”) is separated into components as a result of differential distribution of the analytes, as they flow around or over a stationary liquid or solid phase (“stationary phase”), between said mobile phase and said stationary phase. The stationary phase may be usually a finely divided solid, a sheet of filter material, or a thin film of a liquid on the surface of a solid, or the like. Chromatography is also widely applicable for the separation of chemical compounds of biological origin, such as, e.g., amino acids, proteins, fragments of proteins or peptides, etc.

Chromatography as used herein may be preferably columnar (i.e., wherein the stationary phase is deposited or packed in a column), preferably liquid chromatography, and yet more preferably H PLC. While particulars of chromatography are well known in the art, for further guidance see, e.g., Meyer M., 1998, ISBN: 047198373X, and “Practical HPLC Methodology and Applications”, Bidlingmeyer, B. A., John Wiley & Sons Inc., 1993.

Exemplary types of chromatography include, without limitation, high-performance liquid chromatography (HPLC), normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immuno-affinity, immobilised metal affinity chromatography, and the like.

In an embodiment, chromatography, including single-, two- or more-dimensional chromatography, may be used as a peptide fractionation method in conjunction with a further peptide analysis method, such as for example, with a downstream mass spectrometry analysis as described elsewhere in this specification.

Further peptide or polypeptide separation, identification or quantification methods may be used, optionally in conjunction with any of the above described analysis methods, for measuring biomarkers in the present disclosure. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

The various aspects and embodiments taught herein may further rely on comparing the quantity of the ER-alpha polypeptide as taught herein or one or more of its post-translationally modified forms as defined herein, measured in samples with reference values of the quantity of said polypeptide, wherein said reference values represent known predictions, diagnoses and/or prognoses of ER-alpha related cancers, more specifically ER-alpha-positive breast cancer.

For example, distinct reference values may represent the prediction of a risk (e.g., an abnormally elevated risk) of having ER-alpha related cancers, more specifically ER-alpha-positive breast cancer vs. the prediction of no or normal risk of having ER-alpha related cancers, more specifically ER-alpha-positive breast cancer. In another example, distinct reference values may represent predictions of differing degrees of risk of having ER-alpha related cancers, more specifically ER-alpha-positive breast cancer.

In a further example, distinct reference values can represent the diagnosis of ER-alpha related cancers, more specifically ER-alpha-positive breast cancer vs. the diagnosis of no ER-alpha related cancers, more specifically ER-alpha-positive breast cancer (such as, e.g., the diagnosis of healthy, or recovered from ER-alpha related cancers, more specifically ER-alpha-positive breast cancer, etc.). In another example, distinct reference values may represent the diagnosis of ER-alpha related cancers, more specifically ER-alpha-positive breast cancer of varying severity.

In a yet another example, distinct reference values may represent a good prognosis for ER-alpha related cancers, more specifically ER-alpha-positive breast cancer vs. a poor prognosis for ER-alpha related cancers, more specifically ER-alpha-positive breast cancer. In a further example, distinct reference values may represent varyingly favourable or unfavourable prognoses for ER-alpha related cancers, more specifically ER-alpha-positive breast cancer.

Such comparison may generally include any means to determine the presence or absence of at least one difference and optionally of the size of such different between values or profiles being compared. A comparison may include a visual inspection, an arithmetical or statistical comparison of measurements. Such statistical comparisons include, but are not limited to, applying a rule. If the values or biomarker profiles comprise at least one standard, the comparison to determine a difference in said values or biomarker profiles may also include measurements of these standards, such that measurements of the biomarker are correlated to measurements of the internal standards.

Reference values for the quantity of the ER-alpha polypeptide as taught herein or one or more of its post-translationally modified forms, may be established according to known procedures previously employed for other biomarkers.

For example, a reference value of the quantity of the ER-alpha polypeptide as taught herein or one or more of its post-translationally modified forms for a particular prediction, diagnosis and/or prognosis of ER-alpha related cancers, more specifically ER-alpha-positive breast cancer may be established by determining the quantity of the ER-alpha polypeptide as taught herein or one or more of its post-translationally modified forms in sample(s) from one individual or from a population of individuals characterised by said particular prediction, diagnosis and/or prognosis of ER-alpha related cancers, more specifically ER-alpha-positive breast cancer (i.e., for whom said prediction, diagnosis and/or prognosis of ER-alpha related cancers, more specifically ER-alpha-positive breast cancer holds true). Such population may comprise without limitation ≧2, ≧10, ≧100, or even several hundreds or more individuals.

Hence, by means of an illustrative example, reference values of the quantity of the ER-alpha polypeptide as taught herein or one or more of its post-translationally modified forms for the diagnoses of ER-alpha related cancers, more specifically ER-alpha-positive breast cancer vs. no cancer, may be established by determining the quantity of the ER-alpha polypeptide as taught herein or one or more of its post-translationally modified forms in sample(s) from one individual or from a population of individuals diagnosed (e.g., based on other adequately conclusive means, such as, for example, clinical signs and symptoms, imaging, mammography, etc.) as, respectively, having or not having ER-alpha related cancers.

In an embodiment, reference value(s) as intended herein may convey absolute quantities of the ER-alpha polypeptide as taught herein or one or more of its post-translationally modified forms. In another embodiment, the quantity of the ER-alpha polypeptide as taught herein or one or more of its post-translationally modified forms in a sample from a tested subject may be determined directly relative to the reference value (e.g., in terms of increase or decrease, or fold-increase or fold-decrease). Advantageously, this may allow to compare the quantity of the ER-alpha polypeptide as taught herein or one or more of its post-translationally modified forms in the sample from the subject with the reference value (in other words to measure the relative quantity of the ER-alpha polypeptide as taught herein or one or more of its post-translationally modified forms in the sample from the subject vs the reference value) without the need to first determine the respective absolute quantities of the ER-alpha polypeptide as taught herein or one or more of its post-translationally modified forms.

In an embodiment the present methods may include a step of establishing such reference value(s). In an embodiment, the present kits and devices may include means for establishing a reference value of the quantity of the ER-alpha polypeptide as taught herein or one or more of its post-translationally modified forms for a particular prediction, diagnosis and/or prognosis of ER-alpha related cancers, more specifically ER-alpha-positive breast cancer. Such means may for example comprise one or more samples (e.g., separate or pooled samples) from one or more individuals characterised by said particular prediction, diagnosis and/or prognosis of ER-alpha related cancers, more specifically ER-alpha-positive breast cancer.

The various aspects and embodiments taught herein may further entail finding a deviation or no deviation between the quantity of the ER-alpha polypeptide as taught herein or one or more of its post-translationally modified forms measured in a sample from a subject and a given reference value.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD, or ±1×SE or ±2×SE). Deviation may also refer to a value falling outside of a reference range defined by values in a given population (for example, outside of a range which comprises ≧40%, ≧50%, ≧60%, ≧70%, ≧75% or ≧80% or ≧85% or ≧90% or ≧95% or even ≧100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction, diagnosis and/or prognosis methods, e.g., sensitivity and/or specificity of at least 50%, or at least 60%, or at least 70%, or at least 80%, or at least 85%, or at least 90%, or at least 95%.

For example, in an embodiment, an elevated quantity of the ER-alpha polypeptide as taught herein or one or more of its post-translationally modified forms in the sample from the subject—preferably at least about 1.1-fold elevated, or at least about 1.2-fold elevated, more preferably at least about 1.3-fold elevated, even more preferably at least about 1.4-fold elevated, yet more preferably at least about 1.5-fold elevated, such as between about 1.1-fold and 3-fold elevated or between about 1.5-fold and 2-fold elevated—compared to a reference value representing the prediction or diagnosis of no ER-alpha related cancers, more specifically ER-alpha-positive breast cancer or representing a good prognosis for ER-alpha related cancers, more specifically ER-alpha-positive breast cancer indicates that the subject has or is at risk of having ER-alpha-related disease, more specifically ER-alpha-positive breast cancer or indicates a poor prognosis for ER-alpha related cancers, more specifically ER-alpha-positive breast cancer in the subject.

When a deviation is found between the quantity of the ER-alpha polypeptide as taught herein or one or more of its post-translationally modified forms in a sample from a subject and a reference value representing a certain prediction, diagnosis and/or prognosis of ER-alpha-related disease, more specifically ER-alpha-positive breast cancer, said deviation is indicative of or may be attributed to the conclusion that the prediction, diagnosis and/or prognosis of ER-alpha related cancers, more specifically ER-alpha-positive breast cancer in said subject is different from that represented by the reference value.

When no deviation is found between the quantity of the ER-alpha polypeptide as taught herein or one or more of its post-translationally modified forms in a sample from a subject and a reference value representing a certain prediction, diagnosis and/or prognosis of ER-alpha-related diseases, more specifically ER-alpha-positive breast cancer, the absence of such deviation is indicative of or may be attributed to the conclusion that the prediction, diagnosis and/or prognosis of ER-alpha-related diseases, more specifically ER-alpha-positive breast cancer in said subject is substantially the same as that represented by the reference value.

The previously reported synthetic peptide fragment P₂₉₅-T₃₁₁ of ER-alpha seems to be a regulatory element for the activity of said receptor (Gallo et al., 2007, Mol Cell Endocrinol 268:37-49; Gallo et al., 2007, Letters in Drug Design & Discovery 4:346-355). Our studies have indicated that it actually is an auto-inhibitor motif of the receptor, the deletion thereof in the ER-alpha receptor resulting in a constitutive transcription activity of the receptor (Gallo et al., 2007, Mol Cell Endocrinol 268:37-49; Gallo et al., 2007, Letters in Drug Design & Discovery 4:346-355). It thus seems that the receptor has to switch off the repression displayed by this motif in order to become active (Gallo et al., 2007, Letters in Drug Design & Discovery 4:346-355; Jacquot et al., 2008, J Steroid Biochem Mol Biol 104:1-10). In agreement with this principle, we have shown that competition induced with the synthetic ER-alpha-17p peptide results in an increased transcriptional activity of the ER-alpha receptor (Gallo et al., 2007, Mol Cell Endocrinol 268:37-49; Gallo et al., 2007, Letters in Drug Design & Discovery 4:346-355), as well as in an increased proliferation of breast cancer cell lines that are ER-alpha-positive, but not on cell lines that are ER-alpha-negative (Gallo et al., 2007, Mol Cell Endocrinol 268:37-49; Gallo et al., 2008, J Steroid Biochem Mol Biol 109:138-149).

Exposed to estradiol (E₂), the ER-alpha receptor is rapidly ubiquitinilated and degraded by the proteasome (Nawaz et al., 1999, Proc Natl Acad Sci USA 96:1858-1862; El Khissiin et al., 1999, FEBS Lett 448:160-166). The observation that inhibition of the proteasome with e.g. MG-132, lactacystine or the like diminishes ER-alpha-dependent transcription suggests that said degradation is required for assuring an optimal transcription (Nawaz et al., 1999, Proc Natl Acad Sci USA 96:1858-1862; El Khissiin et al., 1999, FEBS Lett 448:160-166; Laïos et al., 2005, J Steroid Biochem Mol Biol 94:347-359; Gallo et al., 2008, Nucl Recept Signal 6:e007). The present invention now has identified the endogenous polypeptide which is responsible for regulating said hypothesized process.

From conditioned medium of MCF-7 human breast cancer cell line, treated with estradiol, the following ER-alpha breakdown product polypeptide was identified in the present invention (SEQ ID NO:2): K268RQRDDGEGRGEVGSAGDMRAANLWPSPLMIKRSKKNSLALSLT311 of the full-length ER-alpha protein (defined in SEQ ID NO:4, encoded by SEQ ID NO:3).

In addition, the inventors could show that said endogenously secreted protein comprises the following post-translational modifications: methylation of K₂₆₈, R₂₆₉, K₃₀₃, oxidation of M₂₈₆, and phosphorylation of S₂₈₂.

The lysine at position 303 is known to be mutated in high grade breast cancers (Fuqua et al., 2000, Cancer Res 60:4026-4029; Herynk et al., 2004, Endocr Rev 25:869-898; Conway et al., 2005, Breast Cancer Res 7:R871-R880). The inventors hypothesize that these modifications take place right before the degradation of the ER-alpha receptor by the proteasome. We therefore believe these modifications have a great impact on the functionality of the receptor and may be of importance in diagnosis of breast cancer.

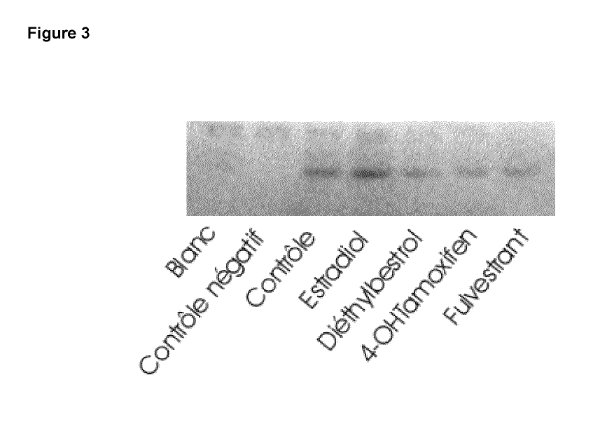

The ER-alpha secretion product (poly)peptide was originally identified by Mass Spectrometry performed on conditioned medium of MCF-7 cells treated with estradiol as explained below in the examples. The results were confirmed by Western-Blot analysis of conditioned medium of MCF-7 cells, after immunoprecipitation with an ER-alpha binding antibody (G-20, Santa-Cruz). The result of this experiment as shown in FIG. 3 demonstrates that estradiol increases the production of the identified endogenous polypeptide identified herein, while 4-OHTamoxifen and Fulvestrant decreases its production. Surprisingly, also diethylstilbestrol slightly reduces the polypeptide production.

The present invention has therefore embarked on a route to identify antibodies that are specific for said endogenous excretion product peptide of the ER-alpha receptor, and its post-translational modifications.

The present invention envisages the study of the effect of inhibitors of the proteasome by using the mechanism discovered herein. A typical assay in this respect comprises the following steps:

a) detecting the presence of the ER-alpha polypeptide identified in the present invention in ER-alpha-positive cell lines and/or in their conditioned media in the presence or absence of a candidate proteasome modulator, wherein a change in the amount of ER-alpha polypeptide indicates the agent is a proteasome modulator. Typically, a change of about 5, 10, 15, 20, 25, 30, 35, 40, 45 50, 55, 60, 65, 70, 75, 80, 85, 90, 95 or even 100% can be comprised under the term “modulating”.

Modulators reducing the amount of ER-alpha polypeptide can be used for treating ER-alpha related disorders such as ER-alpha positive cancers, preferably breast cancer, endometrial cancer, cervical cancer and ovarian cancer.

In the present invention, the MCF-7 breast cancer cell line was used, but this is of course not intended to limit the scope of the invention. Other cell-lines that are ER-alpha-positive (or negative for control experiments) can be used in the respective assays and methods as defined herein. They can be commercially available cell-lines or can be isolated and established from existing human breast cancer biopsies. Non-limiting examples can be a) ER-alpha-positive cell lines: MCF-7, T47D, BT-20, and the like, and b) ER-alpha-negative cell lines: MDA-MB-231, SKBR-3, HS-578T and the like.

The invention provides for a synthetic peptide corresponding to the endogenous peptide as identified herein, optionally with the inclusion of the one or more of the post-translational modifications.

The invention further provides for a method of identifying inhibitors of the ER-alpha receptor comprising the steps of:

a) contacting an ER-alpha positive cell with the peptide according to the invention in the presence and absence of a candidate inhibitor and
b) analyzing the estradiol responsiveness and/or proliferation rate in the presence of estradiol of said cell in the presence and absence of the candidate inhibitor, wherein a reduction of the responsiveness and/or proliferation indicates the candidate indeed is an inhibitor.

The present invention also envisages the study of the effect of ER-alpha polypeptide on endogenous transcriptions of ER-alpha-dependent genes. A typical assay in this respect comprises the following steps:

a) treatment of ER-alpha expressing cells, such as MCF-7 cells, with ER-alpha polypeptide
b) assessment (by western blot, northern blot, RT-PCR) of expression of genes known to be regulated by E₂, typically, progesterone receptors or TFF1 in response to said ER-alpha polypeptide.

The present invention also envisages the study of the effect of ER-alpha polypeptide on the ER-alpha-dependent transcriptions of reporter genes. A typical assay in this respect comprises the following steps:

a) Stable or transient transfection of an ERE-driven reported gene in a cell model (yeast or mammalian cell lines) expressing ER-alpha (natural or ectopic expression)
b) treatment said model with ER-alpha polypeptide
c) assessment of expression of the reporter gene (i.e. luminometry or colorimetry) in the presence of the ER-alpha polypeptide and compare it to the expression of the reporter gene in absence of said ER-alpha polypeptide.

The present invention also envisages the study of the effect of antibodies raised against ER-alpha polypeptide on ER-alpha-dependent transcriptions. A typical assay in this respect comprises the following steps:

a) treatment of ER-alpha expressing cells, such as MCF-7 cells, with antibodies raised against ER-alpha polypeptide.
b) assessment (by western blot, northern blot or RT-PCR) of differences in the expression of genes known to be regulated by E₂, typically, progesterone receptors or TFF1 in the presence or absence of said antibodies.

The present invention also envisages the study of plasmids that are capable of expressing the ER-alpha polypeptide identified in the present invention. A typical assay in this respect comprises the following steps:

a) designing a plasmid coding for this polypeptide
b) transforming and amplifying said plasmid in bacterial cell
c) transfecting said plasmid into human cancer cell lines and assessing the modulation of ER-alpha activity that could be reflected by a decrease of ER-alpha-dependent transcriptions or a decrease of basal and/or E₂-induced cell growth. Typically, a change of about 5, 10, 15, 20, 25, 30, 35, 40, 45 50, 55, 60, 65, 70, 75, 80, 85, 90, 95 or even 100% can be comprised under the term “modulating”.

The present invention provides new methods and tools for the diagnosis of patients suffering from ER-alpha-related cancers or disorders; preferably of breast cancer, ovarian cancer, endometrial cancer or cervical cancer, since the degradation of the ER-alpha provokes the emergence of an estrogenic peptide capable of amplifying hormonal stimuli and thereby the proliferation of e.g. ER-alpha-related cancer cells such as breast cancer cells.

The clinical consequences of the present invention are very important, since it enables us to act directly on the activating pathway of the ER-alpha receptor. By blocking the peptide as defined by the present invention from interacting with the ER-alpha receptor and/or its down stream factors, the ER-alpha activation pathway, leading to proliferation of hormone responsive tumor cells can be inhibited. Capturing said ER-alpha peptide by an inhibitor, an antibody or by vaccination constitutes a new and promising treatment strategy for hormone-dependent cancers such as ER-alpha positive cancer.

The present invention provides methods and compositions directed to ER-alpha-related diseases, and, in some preferred embodiments, to ER-alpha-positive breast cancers. Such ER-alpha-related diseases include, for example: ovarian, endometrial, cervical, lung cancers, head and neck cancers, melanoma, meningiomas, thymomoas and lymphomas. In some specific embodiments, the invention relates to the prevention, treatment, or prevention and treatment of ER-alpha-positive breast cancers in an individual having a risk of developing breast cancer or an individual which has or has not already received treatment for the breast cancer.

Said previous cancer therapy could comprise surgery, chemotherapy, hormone therapy, radiation, or a combination thereof. In a specific embodiment, the individual having already received treatment for the ER-alpha-positive cancer has the cancer in remission. A skilled artisan recognizes that cancer which recurs is not necessarily of the same type as was seen with the original occurrence, and therefore, in a specific embodiment all individuals having had cancer, regardless of the original etiology, are candidates for prevention and treatment with the compositions and methods described herein.

Furthermore, an individual who is at risk for developing cancer or having a recurrence of breast cancer is particularly well-suited to receive therapy with the methods and compositions described herein. A skilled artisan recognizes the multiple risk factors for an individual to develop cancer, including lifestyle and environmental factors, genetic factors, and so forth. Moreover, one skilled in the art recognizes histopathologies and specific mutations which are indicative of an increased risk for developing cancer, particularly with premalignant lesions. Cancer is commonly treated by various combinations of surgery, radiation therapy, chemotherapy, and hormone therapy. Prognosis and selection of therapy may be influenced by the age and menopausal status of the patient, stage of the disease, histologic and nuclear grade of the primary tumor, estrogen-receptor-apha (ER-alpha) and progesterone receptor (PR) status, measures of proliferative capacity, and HER2/neu gene amplification (Simpson et al., 2000).

The invention further provides a composition comprising an isolated ER-alpha-p44 peptide comprising the amino acid sequence of SEQ ID NO.2, preferably consisting of the isolated ER-alpha peptide consisting of the amino acid sequence of SEQ ID NO:2; most preferably comprising one or more of the following post-translational modifications: methylation of K₂₆₈, R₂₆₉, K₃₀₃, and/or phosphorylation of S₂₈₂.

This peptide can be isolated from endogenous protein material or can be synthesised or produced through recombinant expression in a host cell.

The polypeptide in the composition of the present invention can be linked to a carrier molecule such as BSA or KLH, or can be comprised within a lipid composition such as, a lipid particle, a nanocapsule, a liposome, or lipid vesicle. Preferably, said composition comprises a pharmaceutical excipient.

The invention further provides a composition comprising an isolated nucleic acid molecule (e.g. defined by SEQ ID NO:1) encoding the ER-alpha-p44 peptide defined by the amino acid sequence of SEQ ID NO.2, preferably consisting of said nucleic acid molecule. Said nucleic acid molecule or polynucleotide can furthermore be present in the fomr of DNA, cDNA, RNA, mRNA, or in a suitable vector for recombinant expression in a host cell, such as a bacterium, yeast, mammalian or human cell.

The composition according to the present invention can be used in vaccination against tumors and/or cancers. The present invention thus provides for the use of the composition according to the invention for use as a vaccine and/or for use in vaccination. In a preferred embodiment, said vaccination is against human ER-positive cancers, more preferably breast cancers, ovarian cancer, endometrial cancers, or cervical cancers.

The composition in accordance with the invention can be for use in preparing a vaccine for preventing or treating tumors and/or cancers or for use in preparing a cancer inhibitor.

The invention further provides for the use of a composition in accordance with the invention in the manufacture of a medicament or vaccine for use in treating tumors and/or cancers, preferably breast cancer.

The invention further provides for the use of a nucleic acid according to the invention or a peptide according to the invention for use in vaccination.

Vaccination using a polypeptide is well known in the art. In general, the peptide is typically linked or fused to a carrier molecule such as BSA or KLH and combined with one or more adjuvants to elicit an immune response.

In case the polynucleotide is used, said nucleic acid is transferred into a host cell, where it expresses the peptide of the invention (e.g. of SEQ ID NO:2), to which an immune response is to be elicited e.g. in order to reduce cancer growth or wherein the host cell delivers the peptide to its place of apoptotic action. Delivery of said nucleic acid can be done by direct administration of the DNA molecule, or can be done through a bacterial or viral expression system known in the art of gene therapy. This general methodology of DNA vaccination is well known in the art. Recently, direct RNA injection into the lymph nodes of into dendritic cells has been shown to produce antigen-specific antibody producing cells.

The invention thus provides for an isolated nucleic acid that encodes a peptide in accordance with the invention. In a preferred embodiment, said nucleic acid encodes the peptide as defined in SEQ ID NO:2 and is defined by SEQ ID NO:1.

The invention thus provides for a nucleic acid according to the invention for use in preparing a medicament or vaccine for treating tumors and/or cancers or for the use of said nucleic acid in the manufacture of a medicament or vaccine for use in treating tumors and/or cancers.

In addition, the invention provides for a recombinant vector that expresses the ER-alpha-p44 peptide as defined herein. Such a recombinant vector can also be used for gene-therapy, whereby said nucleic acid is transferred to the host and made to express the peptide it is encoding into said host.

The expressed peptide can then possibly be eliciting an immune response in the host, which is targeted to the ER-positive tumor cells.

Said recombinant vector can be for use in preparing a medicament or vaccine for treating cancer or can be used in the manufacture of a medicament or vaccine for use in treating cancer. Viral delivery of RNA or DNA encoding the polypeptide according to the invention for vaccination purposes is also envisaged using known methodologies.

Furthermore, a recombinant host cell expressing the nucleic acid, polypeptide or the recombinant vector as defined herein is envisaged by the present invention, as well as its use in preparing a medicament or vaccine for treating cancer.

Preferably, said host cell is a mammalian cell or cell-line, e.g. a human cell, cancer cell, or cancer cell-line; or alternatively, said cell is a bacterial cell, e.g. an E. coli, Salmonella, or Pseudomonas cell.

The invention further provides for a method of reducing estrogen receptor activity in a cell, comprising providing to said cell an effective inhibitory amount of an inhibitor composition as defined herein.

In a preferred embodiment, said cell is comprised within an animal, and said composition is administered to said animal, preferably, said cell is an animal cell, more preferably said cell is comprised within an animal that has cancer, preferably breast cancer.

The invention further provides for a method of reducing proliferation of a cancer cell, comprising providing to said cancer cell a therapeutically effective amount of an inhibitor of the ER-alpha receptor as defined herein.

In addition, the invention provides for a method of vaccinating a subject comprising the steps of providing to said subject a polypeptide as defined herein, a nucleic acid as defined herein, a recombinant vector as defined herein or a host cell as defined herein.

The invention furthermore provides for a method of treating tumors and/or cancers in an animal, comprising the steps of: administering to said animal a therapeutically-effective amount of a vaccine according to the invention, i.e. comprising a nucleic acid or polypeptide according to the invention.

Preferably, said composition is formulated in a pharmaceutical excipient for administration intravenously, parenterally, orally, topically, or as an inhalant, aerosol or spray. Preferably, said animal is a human.

In an alternative embodiment, the method of treating ER-alpha-positive cancers in an animal further comprises administering at least a second anticancer agent to said animal, such as cyclophosphamide, methotrexate, fluorouracil, adriamycin, tamoxifen, doxorubicin, etoposide, verapamil, podophyllotoxin, and an analog or salt thereof.

The invention furthermore provides for binding molecules that specifically bind to a polypeptide consisting of the amino acid sequence of SEQ ID NO:2. Preferably, said binding molecule specifically binds the polypeptide as defined in SEQ ID NO:2, comprising one or more of the following post-translational modifications: methylation of K₂₆₈, R₂₆₉, K₃₀₃, and/or phosphorylation of S₂₈₂. Examples of binding molecules envisaged hereby are antibodies, monoclonal- or polyclonal antibodies, nanobodies, affybodies, antibody fragments, aptamers, photoaptamers, oligonucleotides, lipocalins, specifically interacting small molecules, Molecular Imprinting Polymers (MIPs), DARPins, ankyrins, specifically interacting proteins, peptidomimetics, biomimetics or peptides, and other molecules that specifically bind to said polypeptide. Both monoclonal, polyclonal or single chain antibodies or fragments thereof that bind one of the biomarkers of the present invention are useful in the methods and kits of the present invention.

Such binding molecules can also act as inhibitors of the ER-alpha function.

The invention thus also provides a pharmaceutical composition comprising an inhibitor of ER-alpha for treating cancer or for use in the manufacturing of a medicament for treating cancer. Preferably, said inhibitor is a binding molecule, specifically binding to the polypeptide of the invention as defined by SEQ ID NO:2, optionally specifically binding to the polypeptide of the invention as defined by SEQ ID NO:2 comprising one or more of the following post-translational modifications: methylation of K₂₆₈, R₂₆₉, K₃₀₃, and/or phosphorylation of S₂₈₂. In a preferred embodiment, said binding molecule is an antibody or fragment thereof.

The antibody according to the present invention can alternatively be attached to a detectable label.

The inhibitors as indicated above can also be used independently, i.e. not in a pharmaceutical composition, for example for the detection of said polypeptide of the invention. The invention thus provides for inhibitors of the ER-alpha receptor protein as such, defined by their ability to bind specifically to the polypeptide as defined herein, preferably defined by SEQ ID NO:2, optionally specifically binding to the polypeptide of the invention as defined by SEQ ID NO:2 comprising one or more of the following post-translational modifications: methylation of K₂₆₈, R₂₆₉, K₃₀₃, and/or phosphorylation of S₂₈₂. In a preferred embodiment, said binding molecule is an antibody or fragment thereof.

Furthermore, an individual who is at risk for developing breast cancer or having a recurrence of breast cancer is particularly well-suited to receive therapy with the methods and compositions described herein. A skilled artisan recognizes the multiple risk factors for an individual to develop breast cancer, including lifestyle and environmental factors, genetic factors, and so forth. Moreover, one skilled in the art recognizes histopathologies and specific mutations which are indicative of an increased risk for developing breast cancer, particularly with premalignant lesions. Breast cancer is commonly treated by various combinations of surgery, radiation therapy, chemotherapy, and hormone therapy. Prognosis and selection of therapy may be influenced by the age and menopausal status of the patient, stage of the disease, histologic and nuclear grade of the primary tumor, estrogen-receptor (ER) and progesterone-receptor (PR) status, measures of proliferative capacity, and HER2/neu gene amplification (Simpson et al., 2000).

Thus, in an object of the present invention there is a method of preventing development or proliferation of one or more cancer cells in an individual comprising administering to the individual the vaccine or composition comprising the ER-alpha-44p peptide according to the present invention or a nucleic acid molecule encoding the latter, optionally in combination with an adjuvant.

In essence, the composition or vaccine as defined herein is used to elicit an immune-response in the subject under treatment, towards the degradation product/peptide of the ER-alpha protein or fragment thereof as defined herein, with the intention of inhibiting the proliferation-inducing activity of said ER-alpha protein or fragment thereof on cancer cells. As is shown in the examples, both the short synthetic ER-alpha-17p peptide (SEQ ID NO: 29) and the long naturally occurring ER-alpha-44p polypeptide (SEQ ID NO: 2) are capable of inducing proliferation of cancer cells. The invention should therefore be seen as envisaging targeting the 44 amino acid polypeptide as well as any fragment thereof comprising at least the 17p peptide sequence. Any one of these fragment will yield an immune response towards a polypeptide comprising at least the 17p active fragment.

The present invention provides new methods and tools for the diagnosis of patients suffering from estrogen-receptor-alpha-related cancers or disorders, since the degradation of the ER-alpha provokes the emergence of an estrogenic peptide capable of amplifying hormonal stimuli and thereby the proliferation of e.g. estrogen-receptor-alpha-related cancer cells such as ER-positive breast, ovarian, endometrial, or cervical cancer cells.

The clinical consequences of the present invention are very important, since it enables us to act directly on the activating pathway of the ER-alpha receptor. By blocking the peptide as defined by the present invention from interacting with the ER-alpha receptor and/or it's down stream factors, the ER-alpha activation pathway, leading to proliferation of hormone responsive tumor cells can be inhibited. Capturing said ER-alpha-peptide by an inhibitor, an antibody or by vaccination constitutes a new and promising treatment strategy for hormone-dependent cancers such as ER-alpha positive breast cancer.

The present invention provides diagnostic methods directed to estrogen receptor-alpha positive (ER-alpha positive) cancers, and, in some preferred embodiments, to ER positive breast cancers. Such ER-alpha positive cancers include, for example: ovarian, endometrial, cervical, lung cancers, head and neck cancers, melanoma, meningiomas, thymomoas and lymphomas. In some specific embodiments, the invention relates to the diagnosis of ER-alpha positive breast cancers in an individual having a risk of developing breast cancer or an individual which has or has not already received treatment for the breast cancer.

Said previous breast cancer therapy could comprise surgery, chemotherapy, radiation, or a combination thereof. In a specific embodiment, the individual having already received treatment for the ER-alpha positive breast cancer has the cancer in remission. A skilled artisan recognizes that breast cancer which recurs is not necessarily of the same type as was seen with the original occurrence, and therefore, in a specific embodiment all individuals having had breast cancer, regardless of the original etiology, are candidates for prevention and treatment with the compositions and methods described herein.

In an additional object of the present invention, there is a method of screening for anti-cancer agents or compounds comprising the steps of:

a) contacting a cancer cell with the ER-alpha polypeptide identified in the present invention,
b) contacting said cells of step a with a candidate anti-cancer agent or compound and measuring the proliferation of said cell in presence and absence of said candidate agent, wherein a decrease of the cell proliferation in the presence of said candidate anti-cancer agent indicates it is an anti-cancer agent, wherein said cancer is of the ER-alpha-dependent-type.

In a specific embodiment, the cell can be in a non-human animal, such as a mouse, rat, rabbit or the like. In a specific embodiment, the method further comprises placing the compound in a pharmacologically acceptable excipient. In a specific embodiment, the test animal can have induced or non induced estrogen receptor alpha positive breast cancer.

The degradation of the ER-alpha receptor generates peptides capable of relaying the hormonal stimulus and thereby promoting the proliferation of hormone-dependent cancer cells.

The detection of such peptides in the serum of patients enables the easy and fast assessment of the presence of estrogen-dependent disorders such as hormone-dependent cancers e.g. breast, endometrial, cervical and ovarian cancer in said subject.

Targeting said peptides using any kind of targeting means such as antibodies, or through vaccination or determining the mode of action of said peptides will enable the development of new cures for said disorders.

In an additional object of the present invention, there is a method of screening for anti-cancer agents or compounds comprising the steps of:

a) contacting an estrogen receptor alpha positive breast cancer cell with the ER-alpha polypeptide identified in the present invention,
b) contacting said cells of step a with a candidate anti-cancer agent or compound and measuring the proliferation of said cell in presence and absence of said candidate agent, wherein a decrease of the cell proliferation in the presence of said candidate anti-cancer agent indicates it is an anti-cancer agent, wherein said cancer is of the ER-alpha-dependent-type.

In a specific embodiment, the cell can be in a non-human animal, such as a mouse, rat, rabbit or the like. In a specific embodiment, the method further comprises placing the compound in a pharmacologically acceptable excipient. In a specific embodiment, the test animal can have induced or non induced estrogen receptor alpha positive breast cancer.

The use of the composition as defined herein can be combined with other known (chemo)therapeutic anti cancer treatments.

Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding molecules, taxol, gemcitabien, navelbine, farnesyl-protein tansferase inhibitors, transplatinum, 5-fluorouracil, vincristin, vinblastin and methotrexate, or any analog or derivative variant of the foregoing. These can be, for example, agents that directly cross-link DNA, agents that intercalate into DNA, and agents that lead to chromosomal and mitotic aberrations by affecting nucleic acid synthesis. Agents that directly cross-link nucleic acids, specifically DNA, are envisaged and are shown herein, to eventuate DNA damage leading to a synergistic antineoplastic combination. Agents such as cisplatin, and other DNA alkylating agents may be used. Agents that damage DNA also include compounds that interfere with DNA replication, mitosis, and chromosomal segregation. Examples of these compounds include adriamycin (also known as doxorubicin), VP-16 (also known as etoposide), verapamil, podophyllotoxin, and the like. Widely used in clinical setting for the treatment of neoplasms, these compounds are administered through bolus injections intravenously at doses ranging 9 from 25-75 mg/m2 at 21 day intervals for adriamycin, to 35-100 mg/m2 for etoposide intravenously or orally.

The degradation of the ER-alpha receptor generates peptides capable of relaying the hormonal stimulus and thereby promoting the proliferation of hormone-dependent cancer cells.

The detection of such peptides in the serum of patients enables the easy and fast assessment of the presence of estrogen-dependent disorders such as hormone-dependent cancers e.g. breast cancer in said subject.

Targeting said peptides using any kind of targeting means such as antibodies, or through vaccination or determining the mode of action of said peptides will enable the development of new cures for said disorders.

The term “sequence identity” as used herein or “identity” in the context of two nucleic acid or amino acid sequences includes reference to the residues in the two sequences which are the same when aligned for maximum correspondence over a specified region. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art.

As used herein, percentage of sequence identity means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

“Sequence identity” has an art-recognized meaning and can be calculated using published techniques. See Computational Molecular Biology, Lesk, ed. (Oxford University Press, 1988), Biocomputing: Informatics And Genome Projects, Smith, ed. (Academic Press, 1993), Computer Analysis Of Sequence Data, Part I, Griffin & Griffin, eds., (Humana Press, 1994), Sequence Analysis In Molecular Biology, Von Heinje ed., Academic Press (1987), Sequence Analysis Primer, Gribskov & Devereux, eds. (Macmillan Stockton Press, 1991), and Carillo & Lipton, SIAM J. Applied Math. 48: 1073 (1988). Methods commonly employed to determine identity or similarity between two sequences include but are not limited to those disclosed in Guide To Huge Computers, Bishop, ed., (Academic Press, 1994) and Carillo & Lipton, supra. Methods to determine identity and similarity are codified in computer programs. Preferred computer program methods to determine identity and similarity between two sequences include but are not limited to the GCG program package (Devereux et al., Nucleic Acids Research 12: 387 (1984)), BLASTN, FASTA (Atschul et al., J. Mol. Biol. 215: 403 (1990)), and FASTDB (Brutlag et al., Comp. App. Biosci. 6: 237 (1990)).

The nucleotide provided by and used in any one of the products, kits or methods of the invention preferably shares at least from about 80% sequence identity, or from about 85% sequence identity, or from about 90% sequence identity, or from about 95% sequence identity, or from about 96% sequence identity, or from about 97% sequence identity, or from about 98% sequence identity, or from about 99% sequence identity, or even about 100% sequence identity to the nucleotide defined by SEQ ID NO:1.

The polypeptide provided by and used in any one of the products, kits or methods of the invention preferably shares at least from about 80% sequence identity, or from about 85% sequence identity, or from about 90% sequence identity, or from about 95% sequence identity, or from about 96% sequence identity, or from about 97% sequence identity, or from about 98% sequence identity, or from about 99% sequence identity, or even about 100% sequence identity to the amino acid sequence defined by SEQ ID NO:2.

### EXAMPLES

The embodiments of the present invention are further illustrated by the following non-limiting examples.

### Example 1

### Detection of a Secreted Endogenous Breakdown Product/Peptide of the ER-Alpha Protein In Vitro

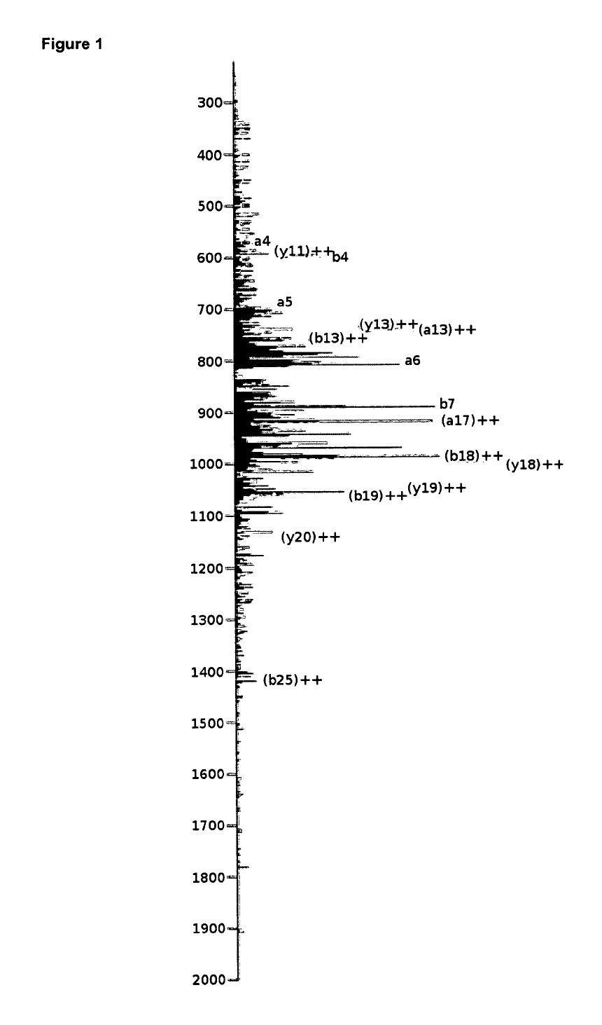

In the present example, the inventors have now been able to detect and characterize an endogenous degradation fragment of the ER-alpha receptor in the medium of breast cancer cell-line MCF-7, treated with E₂, both by Mass-Spectrometry (FIGS. 1 and 2, Tables 1 and 2) and Western-Blotting (FIG. 3).

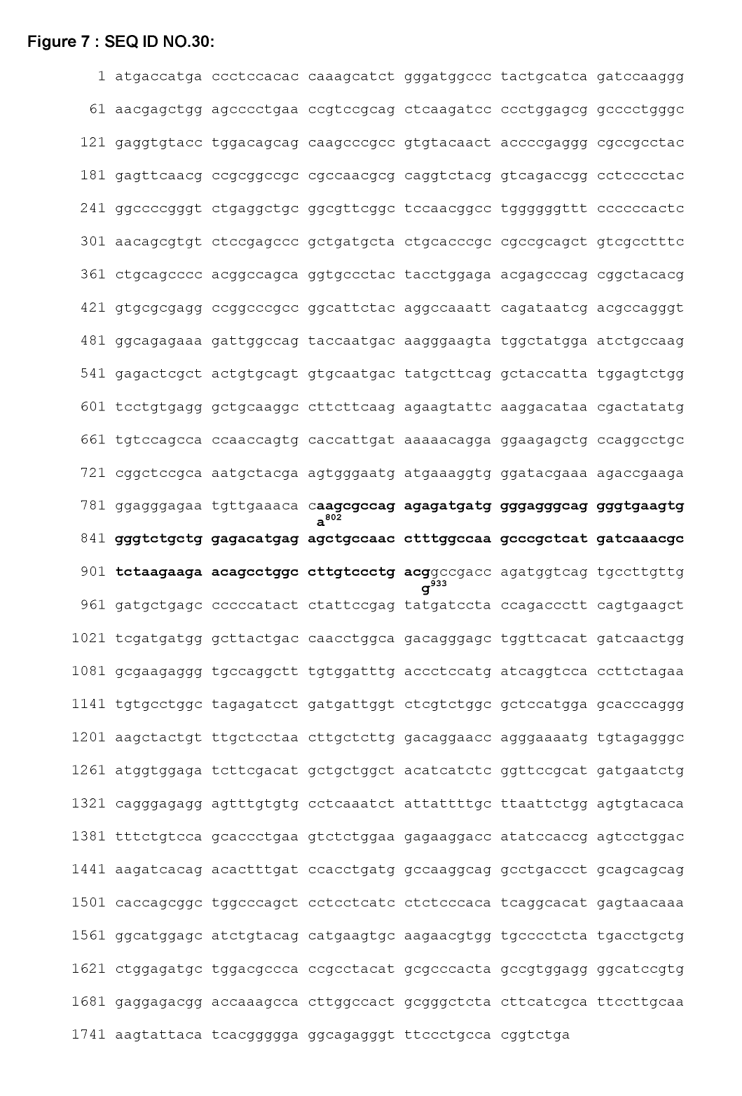

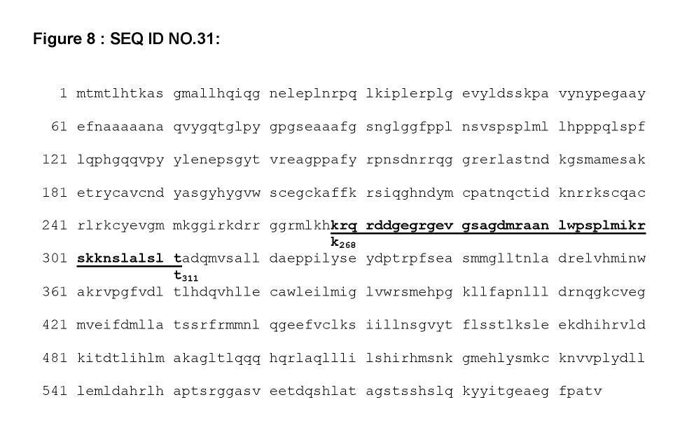

The detected peptide corresponds to the K₂₆₈-T₃₁₁ part of the ER-alpha protein sequence based on the full-length (isoform 1) nucleotide and protein sequence as defined respectively by SEQ ID NO's: 30 and 31 (cf. FIGS. 7 and 8). The degradation product/peptide as such is defined by SEQ ID NO:2 (K₂₆₈RQRDDGEGRGEVGSAGDMRAANLWPSPLMIKRSKKNSLALSLT₃₁₁), as depicted in FIGS. 2 and 8 (bold underlined peptide).

This is a relatively long (44 residues) protein for a proteasome degradation product/peptide, which is unexpected.

The fact that the polypeptide as defined herein is secreted in the medium of MCF-7 cells was unexpected, since the active ER-alpha receptor (i.e. the receptor inducing transcription of its target genes) is mainly situated in cell nucleus. It is therefore interesting to see that a breakdown product/peptide of a nuclear receptor can actually be detected in the culture medium. This finding of course opens the door to diagnostic tests on a specific type of samples of the subjects under analysis, namely those samples that are in close proximity to or in direct contact to tumor or cancer tissue.

At this point, we did not detect the peptide in conditioned medium of control cells, treated or not with E₂ by mass spectrometry, although it may be present. In any case, the fact that the polypeptide of the invention is not detectable or at least much less abundant in normal (i.e. non-tumour) cells, indicates the usefulness of the ER-alpha polypeptide of the invention in the diagnosis of cancers such as breast cancer.

The specific post-translational modifications that were found in the ER-alpha polypeptide as defined herein are also unexpected and might be part of a signaling cascade to trigger the ubiquitination for ER-alpha activation and degradation.

The detected peptide has post-translational modifications that were not reported previously for the native receptor (FIG. 2). The lysines 268 and 303, previously described as being subject to acetylation (Popov et al., 2007, Steroids 72:221-230) are seen in this endogenous peptide as methylated as well as the arginine 269. On the other hand, the serine 282 is phosphorylated.

### Material and Methods

### Preparation of Conditioned Medium:

MCF-7 breast cancer cell line was cultured for 48 hours in EMEM without Phenol-red, comprising 2 mM glutamine, 100 U/ml penicilline and 100 μg/ml streptomycine, supplemented with 10% steroid-free fetal calf serum. After three washing steps with serum/antibiotic-free EMEM without Phenol-red, the cells were cultured for 4 hours in the presence or absence of the E₂ ligand dissolved in serum/antibiotic-free EMEM without Phenol-red, containing only 2 mM glutamine.

### Extraction of Peptides:

After centrifugation, conditioned medium was supplemented with acetonnitrile (ACN) and trifluoro-acetic-acid (TFA) (final concentrations: 5 and 0.5%, respectively). The samples are subsequently passed through C18 micro-columns (Pierce) and peptides are eluted using a solution of H₂O/ACN/TFA:30/70/0.1 v/v/v. The peptides obtained like this were conserved at −20° C. after lyophilisation.

### LC-MS/MS Analysis:

The lyophilised samples are redissolved in a volume of formica cid (FA) of 1%, the peptides are then re-purified on chromatographiques Zip-tip C18 micro-columns (Millipore) before being analysed by Mass Spectrometry. The separations were done using the Ultimate 3000 dual chromatographic system (Dionex). The peptides were loaded onto a pre-column (C18 PepMap100, 5 μm, 100 Å, 300 μm d.i., 5 mm length, Dionex), desalted and concentrated with a solvent A with a flow rate of 15 μl/min during 15 min. They are subsequently eluated to the analytic column with inversed phase polarity (C18 PepMap 100, 3 μm, 100 Å, 75 μm d.i., 15 cm, Dionex) at a flow rate of 220 nl/min. The gradient used is a linear gradient of 0 to 50% of solvent B (ACN/H₂O/AF, 90/10/0.1, v/v/v) in 35 min, followed by an isocratic gradient of 15 min in 100% solvent B and finally 15 min of re-equilibrating of the column in solvent A. The polymers that could be produced by the loading circuit are eliminated due to a purification cartridge (C18 PepMap100, 5 μm, 100 Å, 1 mm d.i., 15 mm de longueur, Dionex). The eluated peptides are detected on the fly on a hybrid mass spectrometer of the LTQ-FTMS (LTQ-FT-ICR, Thermo Fischer) type equipped with a nanospray source with a glass ionizing capillary (New Objective). The instrument is used in the positive mode on the mass gamma of 400-2000Th in mode MS. The resulting data are acquired automatically in mode <<high dynamic>> alternating with a full scan in MS mode of the ICR module, of three SIM scans in the module ICR (+/−5Th around the precursor) in parallel with three times MS/MS in LTQ. After every completed cycle, the 3 most intense peptides are selected (selection window of 4Th) for being fragmented in the trap with an energy of 35%.

### Results

The results of identified peptide are indicated in FIG. 1. The endogenous peptide sequence of the ER-alpha breakdown product/peptide as identified by the present invention is depicted in FIG. 2.

### Example 2

### Immunoprecipitation and Western Blot

In order to provide a proof of principle for establishing a fast and reliable method of detecting the endogenous ER-alpha degradation product/peptide of the invention, we have tested the G20 antibody, which is known to recognize the part of the ER-alpha receptor in a part overlapping with the endogenous peptide as disclosed herein in a Western-blot analysis of conditioned medium of MCF-7 breast cancer cell-lines, treated or not with estradiol or other relevant agents (FIG. 3).

After preclearing with 100 μl (50%) immobilised protein G on agarose beads (UltraLink®, Pierce) (2 hours at 4° C.) conditioned media (or EMEM for the negative control) were incubated with antibody G20 (Santa-Cruz) (except for the negative control) overnight at 4° C. After antibody capturing in 100 μl (50%) immobolised proteine G, the resin is heated to 85° C. for 5 minutes in Laemmli without colorant. The protein samples are subsequently separated on a Tris-Tricine 10% (Invitrogen) gel and the peptides are transferred to nitro-cellulose membranes (15% methanol; semi-dry transfer (BioRad apparatus; 24 V; 0.15 mA, 15 minutes). After blocking with 5% milk for 30 minutes, the proteins are detected with chemiluminescence (primary antibody: G20 (1/500; 2 hours); secondary antibody: anti-rabbit (Pierce; 1/6000; 1 hour).

Indeed, these preliminary results indicate that an antibody such as the anti-ER-alpha G20 antibody (Santa-Cruz), is usable on Western Blot detection of the degradation product/peptide of the ER-alpha receptor as disclosed herein. Although using the same G20 antibody for both capturing and detection could result in some bias of the experiment, our preliminary results indicate (FIG. 3) that the addition of E₂ increases the production of the degradation product/peptide while the addition of either 4-OHTamoxifen or Fulvestrant decrease its production. Interestingly, the addition of diethylstilbestrol seems to decrease the production of the peptide product of the invention as well. The results are indicated in FIG. 3. The peptides were extracted from conditioned medium of MCF-7 cells treated or not (control) with estradiol (10⁻⁸M), diethylstibestrol (10⁻⁸M), 4-OHTamoxifen (10⁻⁷M) or Fulvestrant (10⁻⁷M).

### Example 3

### In Vitro Proliferative Effect the Peptide of the Invention (e.g. SEQ ID NO:2) on MCF-7 Breast Cancer Cell Line

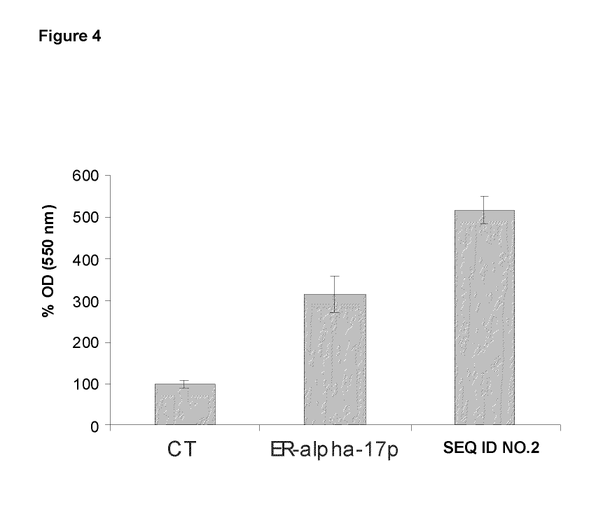

In order to provide a proof of principle that secretion of the ER-alpha breakdown product/peptide described here could relay or amplify the effect of estradiol toward the proliferation of ER-alpha-positive cells, we have treated MCF-7 cells with a synthetic peptide corresponding to this sequence (SEQ ID NO:2) and containing its post-translational modifications (FIG. 4).

ER-alpha-positive MCF-7 breast cancer cell line were seeded in 96-well plates (3000 cells/well). Cells were then treated with or without (control) ER-alpha-17p or the SEQ ID NO:2 peptide both at 10⁻⁵M for 72 hours. Cell growth was measured by crystal violet staining. Briefly, cell cultures were gently washed once with PBS, fixed with 1% glutaraldehyde in PBS (15 min, room temperature) and stained with 0.1% crystal violet (w/v in ddH₂O; 30 min, room temperature). After removal of excess dye by rinsing under gently running tap water, cell-bound crystal violet was extracted with 1% Triton X-100 (v/v in ddH₂O; room temperature, under agitation) and measured by spectrometry at 550 nm.

Results show that a 72 hours treatment of MCF-7 cells with 10⁻⁵M of the peptide SED ID NO:2, in EMEM containing 10% of charcoal-stripped serum, induces a significant increase of their proliferation higher that the control ER-alpha-17p peptide (FIG. 4). This result demonstrate the estrogenic activity of the SED ID NO:2 peptide.

### Example 4

### In Vitro Anti-Proliferative Effect of Antibodies Towards the Peptide of the Invention on MCF-7 Breast Cancer Cell Line

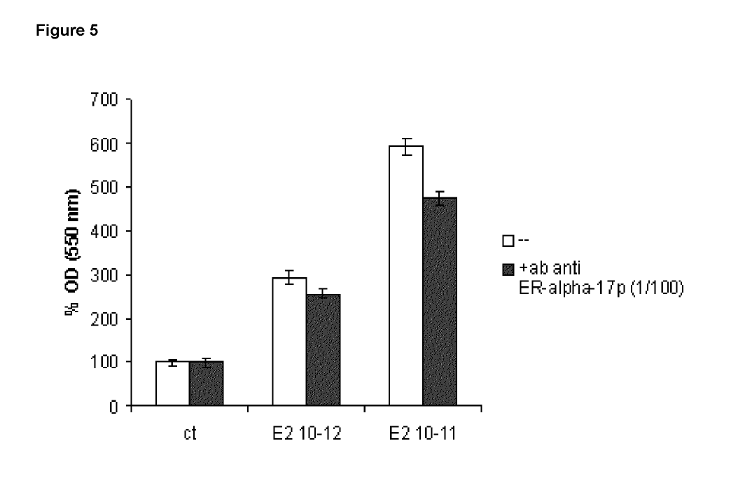

In order to provide a proof of the concept that production of the ER-alpha polypeptide described here is involved in ER-alpha positive breast cancer cells, we assessed the growth of MCF-7 cells in the presence or absence of E₂ and/or an antibody raised against the C-terminal part of this peptide (peptide ER-alpha-17p) (FIG. 5).

ER-alpha-positive MCF-7 breast cancer cell line were seeded in 96-well plates (3000 cells/well). Cells were then treated with E₂ at 10⁻¹¹ or 10⁻¹² M with or without rabbit polyclonal anti-ER-alpha-17p antibody (from Gentaur) with a dilution of 1/100 for 72 hours. Cell growth was measured by crystal violet staining. Briefly, cell cultures were gently washed once with PBS, fixed with 1% glutaraldehyde in PBS (15 min, room temperature) and stained with 0.1% crystal violet (w/v in ddH₂O; 30 min, room temperature). After removal of excess dye by rinsing under gently running tap water, cell-bound crystal violet was extracted with 1% Triton X-100 (v/v in ddH₂O; room temperature, under agitation) and measured by spectrometry at 550 nm.

These preliminary results indicate that an antibody raised against the ER-alpha polypeptide described here may decrease the E₂-induced MCF-7 cell proliferation (FIG. 5).

### Example 5

### In Vitro Effect of the Peptide of the Invention (e.g. SEQ ID NO:2) on ER-Alpha Transcriptional Activity

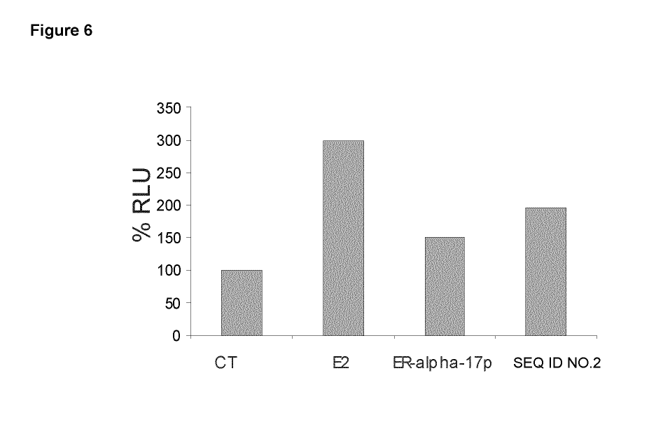

In order to provide a proof of principle that secretion of the ER-alpha breakdown product/peptide described here could relay or amplify the effect of estradiol toward ER-alpha transcriptional activity, we have treated MVLN cells (MCF-7 stably transfected with an ERE-driven luciferase reporter gene; Pons et al., Biotechniques 1990; 9:450-459) with a synthetic peptide corresponding to this sequence (SEQ ID NO:2) and containing its post-translational modifications in serum free condition (FIG. 6).

MVLN cells were cultured for 48 hours in EMEM containing 10% of charcoal-stripped serum prior treatment with E₂ at 10⁻¹⁰ M or ER-alpha-17p or SEQ ID NO:2 both at 10⁻⁵M for. After 6 h treatment in serum free condition, cells were washed 2 times with PBS. Luciferase activity was then measured in cell lysates by luminometry using Luciferase Assay System (Promega) according to manufacturer instructions.

Preliminary results show that a 6 hours treatment of MVLN cells with 10⁻⁵M of the peptide SED ID NO:2, induces a significant increase of the ER-alpha transcriptional activity higher than the control ER-alpha-17p peptide (FIG. 6).

### Example 6

### Analysis of Immunogenicity of Fragments of the Identified Endogenous ER-Alpha Degradation Peptide

In order to develop antibodies that can specifically detect the endogenous peptide identified according to the invention or its fragments, the hydrophillicity, accessibility and antigenicity of said peptide has been evaluated and several interesting potential epitopes have been identified, listed in the tables below. Hence, according to in silico analysis carried out by using the antibody epitope prediction tool available on the immuneepitope.org website, several examples of immunogenic fragments of the KRQRDDGEGRGEVGSAGDMRAANLWPSPLMIKRSKKNSLALSLT peptide are given hereunder:

According to hydrophilicity (Parker hydropilicity algorithm):

### Seven Aminoacid Peptides:

| SEQ | | |
|---|---|---|
| ID | | |
| No. | Peptide | Score |
| 32 | QRDDGEG | 7.057 |
| 33 | DDGEGRG | 7.014 |
| 34 | RQRDDGE | 6.843 |
| 35 | RDDGEGR | 6.800 |
| 36 | DGEGRGE | 6.700 |
| 37 | KRQRDDG | 6.543 |
| 38 | KRSKKNS | 5.900 |
| 39 | EVGSAGD | 4.871 |
| 40 | GEGRGEV | 4.743 |
| 41 | EGRGEVG | 4.743 |
| 42 | GRGEVGS | 4.557 |
| 43 | GSAGDMR | 4.286 |
| 44 | GEVGSAG | 4.257 |
| 45 | RGEVGSA | 4.043 |
| 46 | GDMRAAN | 3.843 |
| 47 | IKRSKKN | 3.829 |
| 48 | SAGDMRA | 3.771 |
| 49 | RSKKNSL | 3.771 |
| 50 | SKKNSLA | 3.471 |
| 51 | VGSAGDM | 3.157 |
| 52 | AGDMRAA | 3.143 |
| 53 | MIKRSKK | 2.229 |
| 54 | DMRAANL | 1.714 |
| 55 | KNSLALS | 1.343 |
| 56 | KKNSLAL | 1.229 |
| 57 | LMIKRSK | 0.100 |
| 58 | AANLWPS | 0.086 |
| 59 | ANLWPSP | 0.086 |

### Eight Aminoacid Peptides:

| SEQ | | |
|---|---|---|
| ID | | |
| No. | Peptide | Score |
| 60 | DDGEGRGE | 7.113 |
| 61 | KRQRDDGE | 6.700 |
| 62 | RQRDDGEG | 6.700 |
| 63 | QRDDGEGR | 6.700 |
| 64 | RDDGEGRG | 6.663 |
| 65 | DGEGRGEV | 5.400 |
| 66 | GEVGSAGD | 4.975 |
| 67 | EGRGEVGS | 4.962 |
| 68 | GEGRGEVG | 4.862 |
| 69 | GRGEVGSA | 4.250 |
| 70 | RGEVGSAG | 4.250 |
| 71 | IKRSKKNS | 4.162 |
| 72 | GSAGDMRA | 4.012 |
| 73 | KRSKKNSL | 4.012 |
| 74 | EVGSAGDM | 3.738 |
| 75 | AGDMRAAN | 3.625 |
| 76 | SAGDMRAA | 3.563 |
| 77 | RSKKNSLA | 3.562 |
| 78 | VGSAGDMR | 3.288 |
| 79 | MIKRSKKN | 2.825 |
| 80 | GDMRAANL | 2.213 |
| 81 | SKKNSLAL | 1.888 |
| 82 | KKNSLALS | 1.888 |
| 83 | LMIKRSKK | 0.800 |
| 84 | RAANLWPS | 0.600 |
| 85 | SPLMIKRS | 0.450 |
| 86 | PLMIKRSK | 0.350 |
| 87 | AANLWPSP | 0.337 |
| 88 | DMRAANLW | 0.250 |
| 89 | KNSLALSL | 0.025 |

### Nine Aminoacid Peptides:

| SEQ | | |
|---|---|---|
| ID | | |
| No. | Peptide | Score |
| 90 | RDDGEGRGE | 6.789 |
| 91 | KRQRDDGEG | 6.589 |
| 92 | QRDDGEGRG | 6.589 |
| 93 | RQRDDGEGR | 6.422 |
| 94 | DDGEGRGEV | 5.911 |
| 95 | DGEGRGEVG | 5.433 |
| 96 | GEGRGEVGS | 5.044 |
| 97 | RGEVGSAGD | 4.889 |
| 98 | EGRGEVGSA | 4.644 |
| 99 | GRGEVGSAG | 4.411 |
| 100 | GEVGSAGDM | 3.956 |
| 101 | SAGDMRAAN | 3.944 |
| 102 | GSAGDMRAA | 3.800 |
| 103 | KRSKKNSLA | 3.800 |
| 104 | EVGSAGDMR | 3.789 |
| 105 | MIKRSKKNS | 3.233 |
| 106 | VGSAGDMRA | 3.156 |
| 107 | IKRSKKNSL | 2.678 |
| 108 | SKKNSLALS | 2.400 |
| 109 | AGDMRAANL | 2.200 |
| 110 | RSKKNSLAL | 2.144 |
| 111 | LMIKRSKKN | 1.489 |
| 112 | SPLMIKRSK | 1.033 |
| 113 | PLMIKRSKK | 0.944 |
| 114 | GDMRAANLW | 0.856 |
| 115 | RAANLWPSP | 0.767 |
| 116 | KKNSLALSL | 0.656 |
| 117 | PSPLMIKRS | 0.633 |
| 118 | KNSLALSLT | 0.600 |
| 119 | DMRAANLWP | 0.456 |

### Ten Aminoacid Peptides:

| SEQ | | |
|---|---|---|
| ID | | |
| No. | Peptide | Score |
| 120 | QRDDGEGRGE | 6.710 |
| 121 | KRQRDDGEGR | 6.350 |
| 122 | RQRDDGEGRG | 6.350 |
| 123 | DDGEGRGEVG | 5.890 |
| 124 | RDDGEGRGEV | 5.740 |
| 125 | DGEGRGEVGS | 5.540 |
| 126 | GRGEVGSAGD | 4.970 |
| 127 | GEGRGEVGSA | 4.750 |
| 128 | EGRGEVGSAG | 4.750 |
| 129 | GSAGDMRAAN | 4.120 |
| 130 | RGEVGSAGDM | 3.980 |
| 131 | GEVGSAGDMR | 3.980 |
| 132 | EVGSAGDMRA | 3.620 |
| 133 | VGSAGDMRAA | 3.050 |
| 134 | SAGDMRAANL | 2.630 |
| 135 | IKRSKKNSLA | 2.620 |
| 136 | RSKKNSLALS | 2.580 |
| 137 | KRSKKNSLAL | 2.500 |
| 138 | LMIKRSKKNS | 1.990 |
| 139 | MIKRSKKNSL | 1.990 |
| 140 | PLMIKRSKKN | 1.550 |
| 141 | SPLMIKRSKK | 1.500 |
| 142 | SKKNSLALSL | 1.240 |
| 143 | PSPLMIKRSK | 1.140 |
| 144 | KKNSLALSLT | 1.110 |
| 145 | DMRAANLWPS | 1.060 |
| 146 | AGDMRAANLW | 0.980 |
| 147 | GDMRAANLWP | 0.980 |
| 148 | MRAANLWPSP | 0.270 |

According to accessibility (Emini Surface Accessibility Prediction):

| SEQ | | |
|---|---|---|
| ID | | |
| No. | Peptide | Score |
| 149 | KRQRDD | 5.400 |
| 150 | KRSKKN | 4.920 |
| 151 | RSKKNS | 3.297 |
| 152 | RQRDDG | 2.672 |
| 153 | QRDDGE | 2.363 |
| 154 | IKRSKK | 2.145 |
| 155 | SKKNSL | 1.388 |
| 156 | RDDGEG | 1.350 |
| 157 | DDGEGR | 1.350 |
| 158 | MIKRSK | 1.061 |
| 159 | KKNSLA | 1.046 |
| 160 | GEGRGE | 0.830 |
| 161 | DGEGRG | 0.800 |
| 162 | DMRAAN | 0.774 |
| 163 | NLWPSP | 0.651 |
| 164 | SAGDMR | 0.632 |
| 165 | EGRGEV | 0.622 |
| 166 | PLMIKR | 0.505 |
| 167 | RGEVGS | 0.482 |
| 168 | AGDMRA | 0.476 |
| 169 | GDMRAA | 0.476 |
| 170 | LMIKRS | 0.438 |
| 171 | KNSLAL | 0.432 |
| 172 | ANLWPS | 0.425 |
| 173 | RAANLW | 0.406 |
| 174 | WPSPLM | 0.401 |
| 175 | MRAANL | 0.382 |
| 176 | GRGEVG | 0.356 |
| 177 | SPLMIK | 0.345 |
| 178 | LWPSPL | 0.334 |
| 179 | AANLWP | 0.321 |
| 180 | GSAGDM | 0.319 |
| 181 | NSLALS | 0.289 |
| 182 | PSPLMI | 0.267 |
| 183 | GEVGSA | 0.248 |
| 184 | EVGSAG | 0.248 |
| 185 | VGSAGD | 0.239 |
| 186 | LALSLT | 0.160 |
| 187 | SLALSL | 0.148 |

According to antigenicity (Kolaskar & Tongaonkar Antigenicity Algorithm)

| SEQ | | |
|---|---|---|
| ID | | |
| No. | Peptide | Score |
| 188 | SLALSLT | 1.107 |
| 189 | NSLALSL | 1.088 |
| 190 | LWPSPLM | 1.051 |
| 191 | NLWPSPL | 1.044 |
| 192 | PSPLMIK | 1.043 |
| 193 | KNSLALS | 1.042 |
| 194 | WPSPLMI | 1.037 |
| 195 | KKNSLAL | 1.030 |
| 196 | AANLWPS | 1.018 |
| 197 | ANLWPSP | 1.018 |
| 198 | SPLMIKR | 1.015 |
| 199 | PLMIKRS | 1.015 |
| 200 | RAANLWP | 0.998 |
| 201 | SKKNSLA | 0.996 |
| 202 | LMIKRSK | 0.996 |
| 203 | GEVGSAG | 0.990 |
| 204 | RGEVGSA | 0.990 |
| 205 | EVGSAGD | 0.989 |
| 206 | VGSAGDM | 0.986 |
| 207 | RSKKNSL | 0.969 |
| 208 | MRAANLW | 0.964 |
| 209 | GRGEVGS | 0.963 |
| 210 | DMRAANL | 0.960 |
| 211 | MIKRSKK | 0.950 |
| 212 | AGDMRAA | 0.947 |
| 213 | IKRSKKN | 0.943 |
| 214 | GEGRGEV | 0.940 |
| 215 | EGRGEVG | 0.940 |
| 216 | SAGDMRA | 0.940 |
| 217 | KRSKKNS | 0.923 |
| 218 | GSAGDMR | 0.913 |
| 219 | GDMRAAN | 0.906 |
| 220 | KRQRDDG | 0.900 |
| 221 | QRDDGEG | 0.888 |
| 222 | RQRDDGE | 0.888 |
| 223 | DDGEGRG | 0.868 |
| 224 | RDDGEGR | 0.868 |
| 225 | DGEGRGE | 0.866 |

Antibodies towards these peptides will be developed by standard techniques.

1. A method for diagnosing estrogen receptor alpha positive cancer in a subject comprising the steps of:
a) detecting in a sample of a subject under analysis, the concentration of an endogenous degradation peptide of the estrogen receptor-alpha (ER-alpha) consisting of any one of the sequences defined by SEQ ID NOs:2 to 29, or 32 to 225, wherein the fragment of SEQ ID NO:29, carries a methylated lysine at position K₃₀₃, and
b) comparing the obtained concentration of said peptide in the sample of the subject to a control concentration of said peptide in a healthy subject, wherein an elevated concentration of said peptide indicates the subject may be suffering of an ER-alpha-positive cancer.

2. The method according to claim 1, wherein said endogenous degradation peptide of the ER-alpha is P₂₉₅LMIKRSKK₃₀₃NSLALSLT₃₁₁ carrying a methylated lysine at position 303 (SEQ ID NO:29).

3. The method according to claim 1, wherein said endogenous degradation peptide consists of the sequence of SEQ ID NO:2.

4. The method according to claim 1, wherein said endogenous degradation peptide carries one or more of the following post-translational modifications: methylation of K₂₆₈, R₂₆₉, K₃₀₃, and/or oxidation of M₂₈₆, and/or phosphorylation of S₂₈₂.

5. The method according to claim 1, wherein said ER-alpha-positive cancer is preferably selected from the group consisting of: breast cancer, endometrial cancer, cervical cancer and ovarian cancer, most preferably human breast cancer.

6. The method according to claim 1, wherein the sample is preferably selected from the group consisting of: whole blood, plasma, serum, nipple aspirate, ductal lavage, tumour exudates, tumour cavity fluid, pleural effusion, acsites fluid, fluid surrounding tumour or cancer cells, lymph, any other bodily fluid in close contact with the tumour or cancer.

7. An isolated estrogen receptor-alpha (ER-alpha) polypeptide consisting of any one of the sequences defined by SEQ ID NOs:2 to 29, or 32 to 225, wherein the fragment of SEQ ID NO:29, carries a methylated lysine at position K₃₀₃.

8. The isolated polypeptide according to claim 7, carrying one or more of the following post-translational modifications: methylation of K₂₆₈, R₂₆₉, K₃₀₃, and/or oxidation of M₂₈₆, and/or phosphorylation of S₂₈₂.

9. A composition comprising the isolated ER-alpha polypeptide according to claim 7, wherein said peptide is linked to a carrier molecule such as bovine serum albumine or keyhole limpet hemocyanin, or comprised within a lipid composition such as, a lipid particle, a nanocapsule, a liposome, or lipid vesicle, optionally further comprising a pharmaceutical excipient.

10. The composition according to claim 9, additionally comprising an adjuvant.

11. (canceled)

12. A nucleic acid molecule encoding the polypeptide according to claim 7.

13. The nucleic acid according to claim 12, consisting of a polynucleotide sequence having at least 90% identity to SEQ ID NO.1, more preferably consisting of the polynucleotide sequence of SEQ ID NO: 1.

14. A method of treating a subject having ER-positive cancer or vaccinating against ER-positive cancer comprising administering the nucleic acid molecule according to claim 12 to the subject wherein the cancer is selected from the group consisting of breast cancer, endometrial cancer, cervical cancer and ovarian cancer, most preferably human breast cancer.

15. A recombinant vector that expresses a peptide according to claim 7.

16. A method of treating a subject having ER-positive cancer or vaccinating against ER-positive cancer comprising administering the recombinant vector according to claim 15 to the subject, wherein the cancer is selected from the group consisting of breast cancer, endometrial cancer, cervical cancer and ovarian cancer, most preferably human breast cancer.

17. A host cell comprising a polypeptide according to claim 7, wherein said cell preferably is a mammalian cell, e.g. a human cell, a yeast cell, a bacterial cell, e.g. an E. coli, Salmonella, or Pseudomonas cell.

18. A method of treating a subject having ER-positive cancer or vaccinating against ER-positive cancer comprising administering the host cell according to claim 17 to the subject, wherein the cancer is selected from the group consisting of breast cancer, endometrial cancer, cervical cancer and ovarian cancer, most preferably human breast cancer.

19. A purified binding molecule that specifically binds to the polypeptide according to claim 7, preferably antibodies, monoclonal- or polyclonal antibodies, nanobodies, affybodies, antibody fragments, aptamers, photoaptamers, oligonucleotides, lipocalins, specifically interacting small molecules, Molecular Imprinting Polymers (MIPs), DARPins, ankyrins, specifically interacting proteins, peptidomimetics, biomimetics or peptides, and other molecules that specifically bind to said polypeptide.

20. An immunodetection kit comprising:
a) a binding molecule according to claim 19,
b) a reference value of the amount of corresponding peptide to which said binding molecule specifically binds, in a healthy subject and
c) instructions to compare the amounts of said peptide in a sample of the subject under investigation and in a sample of a healthy subject in order to conclude whether said subject has ER-alpha positive cancer or not.

21. (canceled)

22. A method of treating a subject having ER-alpha-positive cancer, comprising administering to said subject a therapeutically effective amount of the peptide according to claim 7.

23. A method for vaccinating a subject against the occurrence of ER-alpha-positive cancer, comprising administering to said subject a therapeutically effective amount of the peptide according to claim 7.

24. The method according to claim 22, wherein the peptide carries one or more of the following post-translational modifications: methylation of K₂₆₈, R₂₆₉, K₃₀₃, and/or oxidation of M₂₈₆, and/or phosphorylation of S₂₈₂.

25. The method according to claim 23, wherein the peptide carries one or more of the following post-translational modifications: methylation of K₂₆₈, R₂₆₉, K₃₀₃, and/or oxidation of M₂₈₆, and/or phosphorylation of S₂₈₂.

26. The method according to claim 22, wherein said peptide is linked to a carrier molecule such as bovine serum albumine or keyhole limpet hemocyanin, or comprised within a lipid composition such as, a lipid particle, a nanocapsule, a liposome, or lipid vesicle, optionally further comprising a pharmaceutical excipient.

27. The method according to claim 23, wherein said peptide is linked to a carrier molecule such as bovine serum albumine or keyhole limpet hemocyanin, or comprised within a lipid composition such as, a lipid particle, a nanocapsule, a liposome, or lipid vesicle, optionally further comprising a pharmaceutical excipient.


SEQUENCE LISTING (225 sequences)

SEQ ID NO: 1 - DNA, 132 bp, Homo Sapiens
aagcgccagagagatgatggggagggcaggggtgaagtggggtctgctggagacatgagagctgccaacctttggccaagcccgctcatgatcaaacgctctaagaagaacagcctggccttgtccctgacg

SEQ ID NO: 2 - protein, 44 aa, Homo Sapiens
KRQRDDGEGRGEVGSAGDMRAANLWPSPLMIKRSKKNSLALSLT

SEQ ID NO: 3 - protein, 43 aa, Homo Sapiens
RQRDDGEGRGEVGSAGDMRAANLWPSPLMIKRSKKNSLALSLT

SEQ ID NO: 4 - protein, 42 aa, Homo Sapiens
QRDDGEGRGEVGSAGDMRAANLWPSPLMIKRSKKNSLALSLT

SEQ ID NO: 5 - protein, 41 aa, Homo Sapiens
RDDGEGRGEVGSAGDMRAANLWPSPLMIKRSKKNSLALSLT

SEQ ID NO: 6 - protein, 40 aa, Homo Sapiens
DDGEGRGEVGSAGDMRAANLWPSPLMIKRSKKNSLALSLT

SEQ ID NO: 7 - protein, 39 aa, Homo Sapiens
DGEGRGEVGSAGDMRAANLWPSPLMIKRSKKNSLALSLT

SEQ ID NO: 8 - protein, 38 aa, Homo Sapiens
GEGRGEVGSAGDMRAANLWPSPLMIKRSKKNSLALSLT

SEQ ID NO: 9 - protein, 37 aa, Homo Sapiens
EGRGEVGSAGDMRAANLWPSPLMIKRSKKNSLALSLT

SEQ ID NO: 10 - protein, 36 aa, Homo Sapiens
GRGEVGSAGDMRAANLWPSPLMIKRSKKNSLALSLT

SEQ ID NO: 11 - protein, 35 aa, Homo Sapiens
RGEVGSAGDMRAANLWPSPLMIKRSKKNSLALSLT

SEQ ID NO: 12 - protein, 34 aa, Homo Sapiens
GEVGSAGDMRAANLWPSPLMIKRSKKNSLALSLT

SEQ ID NO: 13 - protein, 33 aa, Homo Sapiens
EVGSAGDMRAANLWPSPLMIKRSKKNSLALSLT

SEQ ID NO: 14 - protein, 32 aa, Homo Sapiens
VGSAGDMRAANLWPSPLMIKRSKKNSLALSLT

SEQ ID NO: 15 - protein, 31 aa, Homo Sapiens
GSAGDMRAANLWPSPLMIKRSKKNSLALSLT

SEQ ID NO: 16 - protein, 30 aa, Homo Sapiens
SAGDMRAANLWPSPLMIKRSKKNSLALSLT

SEQ ID NO: 17 - protein, 29 aa, Homo Sapiens
AGDMRAANLWPSPLMIKRSKKNSLALSLT

SEQ ID NO: 18 - protein, 28 aa, Homo Sapiens
GDMRAANLWPSPLMIKRSKKNSLALSLT

SEQ ID NO: 19 - protein, 27 aa, Homo Sapiens
DMRAANLWPSPLMIKRSKKNSLALSLT

SEQ ID NO: 20 - protein, 26 aa, Homo Sapiens
MRAANLWPSPLMIKRSKKNSLALSLT

SEQ ID NO: 21 - protein, 25 aa, Homo Sapiens
RAANLWPSPLMIKRSKKNSLALSLT

SEQ ID NO: 22 - protein, 24 aa, Homo Sapiens
AANLWPSPLMIKRSKKNSLALSLT

SEQ ID NO: 23 - protein, 23 aa, Homo Sapiens
ANLWPSPLMIKRSKKNSLALSLT

SEQ ID NO: 24 - protein, 22 aa, Homo Sapiens
NLWPSPLMIKRSKKNSLALSLT

SEQ ID NO: 25 - protein, 21 aa, Homo Sapiens
LWPSPLMIKRSKKNSLALSLT

SEQ ID NO: 26 - protein, 20 aa, Homo Sapiens
WPSPLMIKRSKKNSLALSLT

SEQ ID NO: 27 - protein, 19 aa, Homo Sapiens
PSPLMIKRSKKNSLALSLT

SEQ ID NO: 28 - protein, 18 aa, Homo Sapiens
SPLMIKRSKKNSLALSLT

SEQ ID NO: 29 - protein, 17 aa, Homo Sapiens
  MISC_FEATURE (9)..(9): Methyl modification
PLMIKRSKKNSLALSLT

SEQ ID NO: 30 - DNA, 1788 bp, Homo Sapiens
atgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctga

SEQ ID NO: 31 - protein, 595 aa, Homo Sapiens [sequence omitted]

SEQ ID NO: 32 - protein, 7 aa, Artificial Sequence
  Chemically synthesized Peptide
QRDDGEG

SEQ ID NO: 33 - protein, 7 aa, Artificial Sequence
  Chemically synthesized Peptide
DDGEGRG

SEQ ID NO: 34 - protein, 7 aa, Artificial Sequence
  Chemically synthesized Peptide
RQRDDGE

SEQ ID NO: 35 - protein, 7 aa, Artificial Sequence
  Chemically synthesized peptide
RDDGEGR

SEQ ID NO: 36 - protein, 7 aa, Artificial Sequence
  Chemically synthesized Peptide
DGEGRGE

SEQ ID NO: 37 - protein, 7 aa, Artificial Sequence
  Chemically synthesized Peptide
KRQRDDG

SEQ ID NO: 38 - protein, 7 aa, Artificial Sequence
  Chemically synthesized Peptide
KRSKKNS

SEQ ID NO: 39 - protein, 7 aa, Artificial Sequence
  Chemically synthesized Peptide
EVGSAGD

SEQ ID NO: 40 - protein, 7 aa, Artificial Sequence
  Chemically synthesized Peptide
GEGRGEV

SEQ ID NO: 41 - protein, 7 aa, Artificial Sequence
  Chemically synthesized Peptide
EGRGEVG

SEQ ID NO: 42 - protein, 7 aa, Artificial Sequence
  Chemically synthesized Peptide
GRGEVGS

SEQ ID NO: 43 - protein, 7 aa, Artificial Sequence
  Chemically synthesized Peptide
GSAGDMR

SEQ ID NO: 44 - protein, 7 aa, Artificial Sequence
  Chemically synthesized Peptide
GEVGSAG

SEQ ID NO: 45 - protein, 7 aa, Artificial Sequence
  Chemically synthesized Peptide
RGEVGSA

SEQ ID NO: 46 - protein, 7 aa, Artificial Sequence
  Chemically synthesized Peptide
GDMRAAN

SEQ ID NO: 47 - protein, 7 aa, Artificial Sequence
  Chemically Synthesized Peptide
IKRSKKN

SEQ ID NO: 48 - protein, 7 aa, Artificial Sequence
  Chemically Synthesized Peptide
SAGDMRA

SEQ ID NO: 49 - protein, 7 aa, Artificial Sequence
  Chemically Synthesized Peptide
RSKKNSL

SEQ ID NO: 50 - protein, 7 aa, Artificial Sequence
  Chemically Synthesized Peptide
SKKNSLA

SEQ ID NO: 51 - protein, 7 aa, Artificial Sequence
  Chemically Synthesized Peptide
VGSAGDM

SEQ ID NO: 52 - protein, 7 aa, Artificial Sequence
  Chemically Synthesized Peptide
AGDMRAA

SEQ ID NO: 53 - protein, 7 aa, Artificial Sequence
  Chemically Synthesized Peptide
MIKRSKK

SEQ ID NO: 54 - protein, 7 aa, Artificial Sequence
  Chemically Synthesized Peptide
DMRAANL

SEQ ID NO: 55 - protein, 7 aa, Artificial Sequence
  Chemically Synthesized Peptide
KNSLALS

SEQ ID NO: 56 - protein, 7 aa, Artificial Sequence
  Chemically Synthesized Peptide
KKNSLAL

SEQ ID NO: 57 - protein, 7 aa, Artificial Sequence
  Chemically Synthesized Peptide
LMIKRSK

SEQ ID NO: 58 - protein, 7 aa, Artificial Sequence
  Chemically Synthesized Peptide
AANLWPS

SEQ ID NO: 59 - protein, 7 aa, Artificial Sequence
  Chemically Synthesized Peptide
ANLWPSP

SEQ ID NO: 60 - protein, 8 aa, Artificial Sequence
  Chemically Synthesized Peptide
DDGEGRGE

SEQ ID NO: 61 - protein, 8 aa, Artificial Sequence
  Chemically Synthesized Peptide
KRQRDDGE

SEQ ID NO: 62 - protein, 8 aa, Artificial Sequence
  Chemically Synthesized Peptide
RQRDDGEG

SEQ ID NO: 63 - protein, 8 aa, Artificial Sequence
  Chemically Synthesized Peptide
QRDDGEGR

SEQ ID NO: 64 - protein, 8 aa, Artificial Sequence
  Chemically Synthesized Peptide
RDDGEGRG

SEQ ID NO: 65 - protein, 8 aa, Artificial Sequence
  Chemically Synthesized Peptide
DGEGRGEV

SEQ ID NO: 66 - protein, 8 aa, Artificial Sequence
  Chemically Synthesized Peptide
GEVGSAGD

SEQ ID NO: 67 - protein, 8 aa, Artificial Sequence
  Chemically Synthesized Peptide
EGRGEVGS

SEQ ID NO: 68 - protein, 8 aa, Artificial Sequence
  Chemically Synthesized Peptide
GEGRGEVG

SEQ ID NO: 69 - protein, 8 aa, Artificial Sequence
  Chemically Synthesized Peptide
GRGEVGSA

SEQ ID NO: 70 - protein, 8 aa, Artificial Sequence
  Chemically Synthesized Peptide
RGEVGSAG

SEQ ID NO: 71 - protein, 8 aa, Artificial Sequence
  Chemically Synthesized Peptide
IKRSKKNS

SEQ ID NO: 72 - protein, 8 aa, Artificial Sequence
  Chemically Synthesized Peptide
GSAGDMRA

SEQ ID NO: 73 - protein, 8 aa, Artificial Sequence
  Chemically Synthesized Peptide
KRSKKNSL

SEQ ID NO: 74 - protein, 8 aa, Artificial Sequence
  Chemically Synthesized Peptide
EVGSAGDM

SEQ ID NO: 75 - protein, 8 aa, Artificial Sequence
  Chemically Synthesized Peptide
AGDMRAAN

SEQ ID NO: 76 - protein, 8 aa, Artificial Sequence
  Chemically Synthesized Peptide
SAGDMRAA

SEQ ID NO: 77 - protein, 8 aa, Artificial Sequence
  Chemically Synthesized Peptide
RSKKNSLA

SEQ ID NO: 78 - protein, 8 aa, Artificial Sequence
  Chemically Synthesized Peptide
VGSAGDMR

SEQ ID NO: 79 - protein, 8 aa, Artificial Sequence
  Chemically Synthesized Peptide
MIKRSKKN

SEQ ID NO: 80 - protein, 8 aa, Artificial Sequence
  Chemically Synthesized Peptide
GDMRAANL

SEQ ID NO: 81 - protein, 8 aa, Artificial Sequence
  Chemically Synthesized Peptide
SKKNSLAL

SEQ ID NO: 82 - protein, 8 aa, Artificial Sequence
  Chemically Synthesized Peptide
KKNSLALS

SEQ ID NO: 83 - protein, 8 aa, Artificial Sequence
  Chemically Synthesized Peptide
LMIKRSKK

SEQ ID NO: 84 - protein, 8 aa, Artificial Sequence
  Chemically Synthesized Peptide
RAANLWPS

SEQ ID NO: 85 - protein, 8 aa, Artificial Sequence
  Chemically Synthesized Peptide
SPLMIKRS

SEQ ID NO: 86 - protein, 8 aa, Artificial Sequence
  Chemically Synthesized Peptide
PLMIKRSK

SEQ ID NO: 87 - protein, 8 aa, Artificial Sequence
  Chemically Synthesized Peptide
AANLWPSP

SEQ ID NO: 88 - protein, 8 aa, Artificial Sequence
  Chemically Synthesized Peptide
DMRAANLW

SEQ ID NO: 89 - protein, 8 aa, Artificial Sequence
  Chemically Synthesized Peptide
KNSLALSL

SEQ ID NO: 90 - protein, 9 aa, Artificial Sequence
  Chemically Synthesized Peptide
RDDGEGRGE

SEQ ID NO: 91 - protein, 9 aa, Artificial Sequence
  Chemically Synthesized Peptide
KRQRDDGEG

SEQ ID NO: 92 - protein, 9 aa, Artificial Sequence
  Chemically Synthesized Peptide
QRDDGEGRG

SEQ ID NO: 93 - protein, 9 aa, Artificial Sequence
  Chemically Synthesized Peptide
RQRDDGEGR

SEQ ID NO: 94 - protein, 9 aa, Artificial Sequence
  Chemically Synthesized Peptide
DDGEGRGEV

SEQ ID NO: 95 - protein, 9 aa, Artificial Sequence
  Chemically Synthesized Peptide
DGEGRGEVG

SEQ ID NO: 96 - protein, 9 aa, Artificial Sequence
  Chemically Synthesized Peptide
GEGRGEVGS

SEQ ID NO: 97 - protein, 9 aa, Artificial Sequence
  Chemically Synthesized Peptide
RGEVGSAGD

SEQ ID NO: 98 - protein, 9 aa, Artificial Sequence
  Chemically Synthesized Peptide
EGRGEVGSA

SEQ ID NO: 99 - protein, 9 aa, Artificial Sequence
  Chemically Synthesized Peptide
GRGEVGSAG

SEQ ID NO: 100 - protein, 9 aa, Artificial Sequence
  Chemically Synthesized Peptide
GEVGSAGDM

SEQ ID NO: 101 - protein, 9 aa, Artificial Sequence
  Chemically Synthesized Peptide
SAGDMRAAN

SEQ ID NO: 102 - protein, 9 aa, Artificial Sequence
  Chemically Synthesized Peptide
GSAGDMRAA

SEQ ID NO: 103 - protein, 9 aa, Artificial Sequence
  Chemically Synthesized Peptide
KRSKKNSLA

SEQ ID NO: 104 - protein, 9 aa, Artificial Sequence
  Chemically Synthesized Peptide
EVGSAGDMR

SEQ ID NO: 105 - protein, 9 aa, Artificial Sequence
  Chemically Synthesized Peptide
MIKRSKKNS

SEQ ID NO: 106 - protein, 9 aa, Artificial Sequence
  Chemically Synthesized Peptide
VGSAGDMRA

SEQ ID NO: 107 - protein, 9 aa, Artificial Sequence
  Chemically Synthesized Peptide
IKRSKKNSL

SEQ ID NO: 108 - protein, 9 aa, Artificial Sequence
  Chemically Synthesized Peptide
SKKNSLALS

SEQ ID NO: 109 - protein, 9 aa, Artificial Sequence
  Chemically Synthesized Peptide
AGDMRAANL

SEQ ID NO: 110 - protein, 9 aa, Artificial Sequence
  Chemically Synthesized Peptide
RSKKNSLAL

SEQ ID NO: 111 - protein, 9 aa, Artificial Sequence
  Chemically Synthesized Peptide
LMIKRSKKN

SEQ ID NO: 112 - protein, 9 aa, Artificial Sequence
  Chemically Synthesized Peptide
SPLMIKRSK

SEQ ID NO: 113 - protein, 9 aa, Artificial Sequence
  Chemically Synthesized Peptide
PLMIKRSKK

SEQ ID NO: 114 - protein, 9 aa, Artificial Sequence
  Chemically Synthesized Peptide
GDMRAANLW

SEQ ID NO: 115 - protein, 9 aa, Artificial Sequence
  Chemically Synthesized Peptide
RAANLWPSP

SEQ ID NO: 116 - protein, 9 aa, Artificial Sequence
  Chemically Synthesized Peptide
KKNSLALSL

SEQ ID NO: 117 - protein, 9 aa, Artificial Sequence
  Chemically Synthesized Peptide
PSPLMIKRS

SEQ ID NO: 118 - protein, 9 aa, Artificial Sequence
  Chemically Synthesized Peptide
KNSLALSLT

SEQ ID NO: 119 - protein, 9 aa, Artificial Sequence
  Chemically Synthesized Peptide
DMRAANLWP

SEQ ID NO: 120 - protein, 10 aa, Artificial Sequence
  Chemically Synthesized Peptide
QRDDGEGRGE

SEQ ID NO: 121 - protein, 10 aa, Artificial Sequence
  Chemically Synthesized Peptide
KRQRDDGEGR

SEQ ID NO: 122 - protein, 10 aa, Artificial Sequence
  Chemically Synthesized Peptide
RQRDDGEGRG

SEQ ID NO: 123 - protein, 10 aa, Artificial Sequence
  Chemically Synthesized Peptide
DDGEGRGEVG

SEQ ID NO: 124 - protein, 10 aa, Artificial Sequence
  Chemically Synthesized Peptide
RDDGEGRGEV

SEQ ID NO: 125 - protein, 10 aa, Artificial Sequence
  Chemically Synthesized Peptide
DGEGRGEVGS

SEQ ID NO: 126 - protein, 10 aa, Artificial Sequence
  Chemically Synthesized Peptide
GRGEVGSAGD

SEQ ID NO: 127 - protein, 10 aa, Artificial Sequence
  Chemically Synthesized Peptide
GEGRGEVGSA

SEQ ID NO: 128 - protein, 10 aa, Artificial Sequence
  Chemically Synthesized Peptide
EGRGEVGSAG

SEQ ID NO: 129 - protein, 10 aa, Artificial Sequence
  Chemically Synthesized Peptide
GSAGDMRAAN

SEQ ID NO: 130 - protein, 10 aa, Artificial Sequence
  Chemically Synthesized Peptide
RGEVGSAGDM

SEQ ID NO: 131 - protein, 10 aa, Artificial Sequence
  Chemically Synthesized Peptide
GEVGSAGDMR

SEQ ID NO: 132 - protein, 10 aa, Artificial Sequence
  Chemically Synthesized Peptide
EVGSAGDMRA

SEQ ID NO: 133 - protein, 10 aa, Artificial Sequence
  Chemically Synthesized Peptide
VGSAGDMRAA

SEQ ID NO: 134 - protein, 10 aa, Artificial Sequence
  Chemically Synthesized Peptide
SAGDMRAANL

SEQ ID NO: 135 - protein, 10 aa, Artificial Sequence
  Chemically Synthesized Peptide
IKRSKKNSLA

SEQ ID NO: 136 - protein, 10 aa, Artificial Sequence
  Chemically Synthesized Peptide
RSKKNSLALS

SEQ ID NO: 137 - protein, 10 aa, Artificial Sequence
  Chemically Synthesized Peptide
KRSKKNSLAL

SEQ ID NO: 138 - protein, 10 aa, Artificial Sequence
  Chemically Synthesized Peptide
LMIKRSKKNS

SEQ ID NO: 139 - protein, 10 aa, Artificial Sequence
  Chemically Synthesized Peptide
MIKRSKKNSL

SEQ ID NO: 140 - protein, 10 aa, Artificial Sequence
  Chemically Synthesized Peptide
PLMIKRSKKN

SEQ ID NO: 141 - protein, 10 aa, Artificial Sequence
  Chemically Synthesized Peptide
SPLMIKRSKK

SEQ ID NO: 142 - protein, 10 aa, Artificial Sequence
  Chemically Synthesized Peptide
SKKNSLALSL

SEQ ID NO: 143 - protein, 10 aa, Artificial Sequence
  Chemically Synthesized Peptide
PSPLMIKRSK

SEQ ID NO: 144 - protein, 10 aa, Artificial Sequence
  Chemically Synthesized Peptide
KKNSLALSLT

SEQ ID NO: 145 - protein, 10 aa, Artificial Sequence
  Chemically Synthesized Peptide
DMRAANLWPS

SEQ ID NO: 146 - protein, 10 aa, Artificial Sequence
  Chemically Synthesized Peptide
AGDMRAANLW

SEQ ID NO: 147 - protein, 10 aa, Artificial Sequence
  Chemically Synthesized Peptide
GDMRAANLWP

SEQ ID NO: 148 - protein, 10 aa, Artificial Sequence
  Chemically Synthesized Peptide
MRAANLWPSP

SEQ ID NO: 149 - protein, 6 aa, Artificial Sequence
  Chemically Synthesized Peptide
KRQRDD

SEQ ID NO: 150 - protein, 6 aa, Artificial Sequence
  Chemically Synthesized Peptide
KRSKKN

SEQ ID NO: 151 - protein, 6 aa, Artificial Sequence
  Chemically Synthesized Peptide
RSKKNS

SEQ ID NO: 152 - protein, 6 aa, Artificial Sequence
  Chemically Synthesized Peptide
RQRDDG

SEQ ID NO: 153 - protein, 6 aa, Artificial Sequence
  Chemically Synthesized Peptide
QRDDGE

SEQ ID NO: 154 - protein, 6 aa, Artificial Sequence
  Chemically Synthesized Peptide
IKRSKK

SEQ ID NO: 155 - protein, 6 aa, Artificial Sequence
  Chemically Synthesized Peptide
SKKNSL

SEQ ID NO: 156 - protein, 6 aa, Artificial Sequence
  Chemically Synthesized Peptide
RDDGEG

SEQ ID NO: 157 - protein, 6 aa, Artificial Sequence
  Chemically Synthesized Peptide
DDGEGR

SEQ ID NO: 158 - protein, 6 aa, Artificial Sequence
  Chemically Synthesized Peptide
MIKRSK

SEQ ID NO: 159 - protein, 6 aa, Artificial Sequence
  Chemically Synthesized Peptide
KKNSLA

SEQ ID NO: 160 - protein, 6 aa, Artificial Sequence
  Chemically Synthesized Peptide
GEGRGE

SEQ ID NO: 161 - protein, 6 aa, Artificial Sequence
  Chemically Synthesized Peptide
DGEGRG

SEQ ID NO: 162 - protein, 6 aa, Artificial Sequence
  Chemically Synthesized Peptide
DMRAAN

SEQ ID NO: 163 - protein, 6 aa, Artificial Sequence
  Chemically Synthesized Peptide
NLWPSP

SEQ ID NO: 164 - protein, 6 aa, Artificial Sequence
  Chemically Synthesized Peptide
SAGDMR

SEQ ID NO: 165 - protein, 6 aa, Artificial Sequence
  Chemically Synthesized Peptide
EGRGEV

SEQ ID NO: 166 - protein, 6 aa, Artificial Sequence
  Chemically Synthesized Peptide
PLMIKR

SEQ ID NO: 167 - protein, 6 aa, Artificial Sequence
  Chemically Synthesized Peptide
RGEVGS

SEQ ID NO: 168 - protein, 6 aa, Artificial Sequence
  Chemically Synthesized Peptide
AGDMRA

SEQ ID NO: 169 - protein, 6 aa, Artificial Sequence
  Chemically Synthesized Peptide
GDMRAA

SEQ ID NO: 170 - protein, 6 aa, Artificial Sequence
  Chemically Synthesized Peptide
LMIKRS

SEQ ID NO: 171 - protein, 6 aa, Artificial Sequence
  Chemically Synthesized Peptide
KNSLAL

SEQ ID NO: 172 - protein, 6 aa, Artificial Sequence
  Chemically Synthesized Peptide
ANLWPS

SEQ ID NO: 173 - protein, 6 aa, Artificial Sequence
  Chemically Synthesized Peptide
RAANLW

SEQ ID NO: 174 - protein, 6 aa, Artificial Sequence
  Chemically Synthesized Peptide
WPSPLM

SEQ ID NO: 175 - protein, 6 aa, Artificial Sequence
  Chemically Synthesized Peptide
MRAANL

SEQ ID NO: 176 - protein, 6 aa, Artificial Sequence
  Chemically Synthesized Peptide
GRGEVG

SEQ ID NO: 177 - protein, 6 aa, Artificial Sequence
  Chemically Synthesized Peptide
SPLMIK

SEQ ID NO: 178 - protein, 6 aa, Artificial Sequence
  Chemically Synthesized Peptide
LWPSPL

SEQ ID NO: 179 - protein, 6 aa, Artificial Sequence
  Chemically Synthesized Peptide
AANLWP

SEQ ID NO: 180 - protein, 6 aa, Artificial Sequence
  Chemically Synthesized Peptide
GSAGDM

SEQ ID NO: 181 - protein, 6 aa, Artificial Sequence
  Chemically Synthesized Peptide
NSLALS

SEQ ID NO: 182 - protein, 6 aa, Artificial Sequence
  Chemically Synthesized Peptide
PSPLMI

SEQ ID NO: 183 - protein, 6 aa, Artificial Sequence
  Chemically Synthesized Peptide
GEVGSA

SEQ ID NO: 184 - protein, 6 aa, Artificial Sequence
  Chemically Synthesized Peptide
EVGSAG

SEQ ID NO: 185 - protein, 6 aa, Artificial Sequence
  Chemically Synthesized Peptide
VGSAGD

SEQ ID NO: 186 - protein, 6 aa, Artificial Sequence
  Chemically Synthesized Peptide
LALSLT

SEQ ID NO: 187 - protein, 6 aa, Artificial Sequence
  Chemically Synthesized Peptide
SLALSL

SEQ ID NO: 188 - protein, 7 aa, Artificial Sequence
  Chemically Synthesized Peptide
SLALSLT

SEQ ID NO: 189 - protein, 7 aa, Artificial Sequence
  Chemically Synthesized Peptide
NSLALSL

SEQ ID NO: 190 - protein, 7 aa, Artificial Sequence
  Chemically Synthesized Peptide
LWPSPLM

SEQ ID NO: 191 - protein, 7 aa, Artificial Sequence
  Chemically Synthesized Peptide
NLWPSPL

SEQ ID NO: 192 - protein, 7 aa, Artificial Sequence
  Chemically Synthesized Peptide
PSPLMIK

SEQ ID NO: 193 - protein, 7 aa, Artificial Sequence
  Chemically Synthesized Peptide
KNSLALS

SEQ ID NO: 194 - protein, 7 aa, Artificial Sequence
  Chemically Synthesized Peptide
WPSPLMI

SEQ ID NO: 195 - protein, 7 aa, Artificial Sequence
  Chemically Synthesized Peptide
KKNSLAL

SEQ ID NO: 196 - protein, 7 aa, Artificial Sequence
  Chemically Synthesized Peptide
AANLWPS

SEQ ID NO: 197 - protein, 7 aa, Artificial Sequence
  Chemically Synthesized Peptide
ANLWPSP

SEQ ID NO: 198 - protein, 7 aa, Artificial Sequence
  Chemically Synthesized Peptide
SPLMIKR

SEQ ID NO: 199 - protein, 7 aa, Artificial Sequence
  Chemically Synthesized Peptide
PLMIKRS

SEQ ID NO: 200 - protein, 7 aa, Artificial Sequence
  Chemically Synthesized Peptide
RAANLWP

SEQ ID NO: 201 - protein, 7 aa, Artificial Sequence
  Chemically Synthesized Peptide
SKKNSLA

SEQ ID NO: 202 - protein, 7 aa, Artificial Sequence
  Chemically Synthesized Peptide
LMIKRSK

SEQ ID NO: 203 - protein, 7 aa, Artificial Sequence
  Chemically Synthesized Peptide
GEVGSAG

SEQ ID NO: 204 - protein, 7 aa, Artificial Sequence
  Chemically Synthesized Peptide
RGEVGSA

SEQ ID NO: 205 - protein, 7 aa, Artificial Sequence
  Chemically Synthesized Peptide
EVGSAGD

SEQ ID NO: 206 - protein, 7 aa, Artificial Sequence
  Chemically Synthesized Peptide
VGSAGDM

SEQ ID NO: 207 - protein, 7 aa, Artificial Sequence
  Chemically Synthesized Peptide
RSKKNSL

SEQ ID NO: 208 - protein, 7 aa, Artificial Sequence
  Chemically Synthesized Peptide
MRAANLW

SEQ ID NO: 209 - protein, 7 aa, Artificial Sequence
  Chemically Synthesized Peptide
GRGEVGS

SEQ ID NO: 210 - protein, 7 aa, Artificial Sequence
  Chemically Synthesized Peptide
DMRAANL

SEQ ID NO: 211 - protein, 7 aa, Artificial Sequence
  Chemically Synthesized Peptide
MIKRSKK

SEQ ID NO: 212 - protein, 7 aa, Artificial Sequence
  Chemically Synthesized Peptide
AGDMRAA

SEQ ID NO: 213 - protein, 7 aa, Artificial Sequence
  Chemically Synthesized Peptide
IKRSKKN

SEQ ID NO: 214 - protein, 7 aa, Artificial Sequence
  Chemically Synthesized Peptide
GEGRGEV

SEQ ID NO: 215 - protein, 7 aa, Artificial Sequence
  Chemically Synthesized Peptide
EGRGEVG

SEQ ID NO: 216 - protein, 7 aa, Artificial Sequence
  Chemically Synthesized Peptide
SAGDMRA

SEQ ID NO: 217 - protein, 7 aa, Artificial Sequence
  Chemically Synthesized Peptide
KRSKKNS

SEQ ID NO: 218 - protein, 7 aa, Artificial Sequence
  Chemically Synthesized Peptide
GSAGDMR

SEQ ID NO: 219 - protein, 7 aa, Artificial Sequence
  Chemically Synthesized Peptide
GDMRAAN

SEQ ID NO: 220 - protein, 7 aa, Artificial Sequence
  Chemically Synthesized Peptide
KRQRDDG

SEQ ID NO: 221 - protein, 7 aa, Artificial Sequence
  Chemically Synthesized Peptide
QRDDGEG

SEQ ID NO: 222 - protein, 7 aa, Artificial Sequence
  Chemically Synthesized Peptide
RQRDDGE

SEQ ID NO: 223 - protein, 7 aa, Artificial Sequence
  Chemically Synthesized Peptide
DDGEGRG

SEQ ID NO: 224 - protein, 7 aa, Artificial Sequence
  Chemically Synthesized Peptide
RDDGEGR

SEQ ID NO: 225 - protein, 7 aa, Artificial Sequence
  Chemically Synthesized Peptide
DGEGRGE